(19) United States
(12) Patent Application Publication
(10) Pub. No.: US 2015/0219667 A1
(43) Pub. Date: Aug. 6, 2015

(54) PRE-TRANSPLANT IgG REACTIVITY TO APOPTOTIC CELLS CORRELATES WITH LATE KIDNEY ALLOGRAFT LOSS

(71) Applicant: Emmanuel Zorn, New York, NY (US)

(72) Inventor: Emmanuel Zorn, New York, NY (US)

(73) Assignee: The Trustees of Columbia University in the City of New York, New York, NY (US)

(21) Appl. No.: 14/614,998

(22) Filed: Feb. 5, 2015

Related U.S. Application Data
(60) Provisional application No. 61/935,910, filed on Feb. 5, 2014.

(51) Int. Cl. G01N 33/68; A61M 1/34

(52) U.S. Cl. CPC G01N 33/6854; A61M 1/3496; A61M 2202/0417; A61M 2202/0021; G01N 2800/245; G01N 2800/50

(57) ABSTRACT

It has been discovered that significantly elevated levels of anti-apoptotic cell IgG is an important contributor to and predictor of late graft rejection. Kaplan-Meier survival analysis revealed that patients with high pre-transplant IgG and post-transplant reactivity to apoptotic cells had a significantly increased rate of late graft loss that was apparent after approximately 1 year post-transplant. This association between pre-transplant IgG reactivity to apoptotic cells and graft loss was still significant after excluding patients with high reactivity to HLA, and it was almost exclusively mediated by IgG1 and IgG3 with complement fixing and activating properties. The association between elevated levels of anti-apoptotic cell IgG antibodies and late transplant rejection forms a basis for diagnosing and treating patients at high risk of late transplant rejection.

### CROSS-REFERENCE TO RELATED APPLICATIONS

This application claims benefit of Provisional Appln. 61/935,910, filed Feb. 5, 2014, the entire contents of which are hereby incorporated by reference as if fully set forth herein, under 35 U.S.C. §119(e).

### STATEMENT OF GOVERNMENT INTEREST

The invention was made with government support under grant Contract No. NIH NIDDK DK083352 awarded by the National Institutes of Health. The government has certain rights in the invention.

### BACKGROUND

Early detection of solid organ graft rejection or graft injury is a significant unmet clinical need. Biopsy-based methods have poor sensitivity and high risk of severe complications. For the recipient of a major organ transplant such as a heart, lung, kidney, liver or pancreas, transplantation is often the only realistic chance for mid- and long-term survival in view of the severity of the underlying disease or injury. Today, it is estimated that at least several ten thousand major transplantations are performed every year. The most frequently transplanted organ is the kidney, of which about 25,000 are transplanted per year in the USA alone, followed by the liver, heart, lung, and pancreas. The success of these life-extending procedures has markedly increased over the past decades however; rejection especially late rejection is still a significant threat and real complication.

Acute rejection remains a common and serious post-transplantation complication that is also a risk factor for chronic rejection, a relentlessly progressive process. As the occurrence of acute rejection episodes is the most powerful predictive factor for the later development of chronic rejection in adults and children, noninvasive methods for predicting rejection are needed. Acute renal allograft rejection is currently diagnosed following percutaneous needle core biopsy of the allograft, an invasive biopsy procedure. While increased serum creatinine levels are currently the best surrogate markers of acute kidney rejection, it lacks sensitivity and specificity with respect to predicting rejection. In fact, despite the availability of these diagnostic methods, almost 30% of allograft biopsies performed in renal allograft recipients with stable renal function and an equivalent percentage of allografts successfully treated with anti-rejection drugs reveal authentic histologic features of acute rejection. These occult rejections are unmasked by protocol biopsies but they are unattended by clinical signs such as an increase in serum creatinine levels.

Current procedures to diagnose allograft rejection generally depend upon detection of graft dysfunction and the presence of a mononuclear leukocytic infiltrate. However, the presence of a modest cellular infiltrate is often inconclusive as it can be detected in non-rejecting grafts. Repetitive samplings of the allograft, while ideal from a diagnostic perspective, are invasive and increase morbidity. Therefore there is still a great need for a noninvasive method for early detection of pre-transplant subjects and transplant subjects who are at high risk of transplant rejection.

### SUMMARY

It has been discovered that elevated levels of anti-apoptotic IgG can be used to predict transplant rejection in pre- and post-transplant subjects.

Certain embodiments are directed to method for predicting transplant rejection in a pre-transplant subject or a subject who has had a transplant, by (a) obtaining a biological sample from the subject and a biological sample from a group of healthy control subjects; (b) isolating IgG antibodies from the subject and from at least three control samples; (c) contacting a test population of apoptotic cells with the subject IgG antibodies and contacting at least three control populations of apoptotic cells with the IgG antibodies from each of the control samples, for a time and under conditions that permit the antibodies to bind to the apoptotic cells; and (d) determining the amount of binding of the IgG antibodies to apoptotic cells in the test population and in the control populations, and if the amount of IgG antibody binding in the test population is higher than the median value+2 standard deviation of three control specimens, then determining that the subject is at a high risk of transplant rejection. Any apoptotic cell can be used, including Jurkat cells, 293 Human Embryonic Kidney cells, and endothelial cells including human umbilical cord endothelial cells. In some embodiments determining the amount of binding of the IgG antibodies to apoptotic cells in the test population and in the control populations comprises incubating the test and control apoptotic cells of step (e) with a secondary anti-IgG antibody; and assessing binding of the IgG antibodies to quantitate antibody binding.

The embodiment methods can be used in any pre- or post-transplant subject including those needing or having had a tissue transplant (corneas, bone, tendons, ligaments, heart valves, skin, blood vessels, veins, arteries and hematopoietic stem cell transplants) and organ transplant (pancreas, heart, kidney, lung, liver, bladder and intestine). Appropriate biological samples include blood, plasma, serum or other blood derived products, csf, synovial fluid, bronchioalveolar lavage and ascites. In another embodiment, the subject is given a desensitization treatment if it is determined that the subject is at risk of transplantation rejection, then administering to the subject a desensitization treatment, including Plasmapheresis or administering a therapeutically effective amount of an immunosuppressant drug selected from the group consisting of Bortezomib, cyclosporine, rapamycin, Campath I, thymoglobulin, (rATG), anti-thymocytic antibody, Rituximab, and Gamimune N, dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur, fluocinolone, triaminolone, anecortave acetate, flurometholone, medrysone, IVIG and prednislone.

Other embodiments are directed to kits for prediction of rejection of a transplanted organ or tissue comprising: (a) a biological sample collection device to obtain a serum sample from a subject; (b) a serum sample from each of at least 3 normal control subjects comprising isolated IgG antibodies; (c) and instructions for using the kit and optionally also (d) apoptotic cells capable of binding IgG antibodies.

These and other features, aspects, and advantages of the present invention will become better understood with regard to the following description, appended claims, and accompanying figures.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1. Pre-transplant purified IgG reactivity to apoptotic cells. The purified IgG reactivity to apoptotic Jurkat cells was measured by flow cytometry in samples collected pre-transplant from kidney transplant recipients as well as healthy subjects. Log₂ values of MFI are reported (y-axis). The numbers of samples tested in each group are shown below the box plot. The horizontal bar represents the median value; the bottom and top of each box represent the 25th and 75th percentiles; the lower and upper bars of each box represent the minimum and maximum values.

FIG. 2. Pre-transplant purified IgG reactivity to apoptotic cells and graft loss. (A) Pre-transplant IgG reactivity to apoptotic cells was compared between patients with functioning graft and patients who experienced graft loss and (B) between patients whose graft loss was attributed to AMR and patients with other causes of graft loss. The numbers of samples analyzed in each group are shown below the box plot. The horizontal bar represents the median value; the bottom and top of each box represent the 25th and 75th percentiles; the lower and upper bars of each box represent the minimum and maximum values.

FIG. 3. Pre-transplant purified IgG reactivity to apoptotic cells and graft outcome. Kaplan-Meier cumulative, death censored, graft survival plot analyzing the effect of pre-transplant purified IgG reactivity to apoptotic Jurkat cells (A) below (black line) or above (red line) the median value. (B) below the 1^{st} quartile (black line), between the 1^{st} quartile and the 2^{nd} quartile (blue line), between the 2^{nd} and 3^{rd} quartile (green line) or above the 3^{rd} quartile value (red line). The number of patients at risk is shown below each time point. P value among groups was computed using log-rank (Mantel-Cox) test.

FIG. 4. Kaplan-Meier cumulative graft survival. (A) Kaplan-Meier cumulative, death censored, graft survival plot analyzing the effect of pre-transplant IgG reactivity to HLA class I, HLA class II or MICA above (dot line) or below (solid line) a cutoff MFI value of 1000. (B) Kaplan-Meier cumulative, death censored, graft survival plot analyzing the effect of pre-transplant purified IgG reactivity to apoptotic Jurkat cells above (dotted line) or below (solid line) the median value after exclusion of patients with high reactivity to HLA class I, HLA class II or MICA (MFI>1000). (C) Kaplan-Meier cumulative, death censored, graft survival plot analyzing the effect of pre-transplant IgG reactivity to HLA class I, HLA class II or MICA above (dot line) or below (solid line) a cutoff MFI value of 1000 after exclusion of patients with reactivity to apoptotic cells above the median value. The number of patients at risk is shown below each time point. P value between two groups was computed using log-rank (Mantel-Cox) test.

FIG. 5. Subclasses of serum IgG reactive to apoptotic cells. (A) Example of subclass analysis by flow cytometry of IgG reactive to apoptotic Jurkat cells purified from 4 representative pre-transplant serum specimens, using secondary antibodies specific to IgG1˜IgG4. Filled gray histograms show background reactivity obtained with secondary antibodies alone. IgG1˜IgG4 reactivity to apoptotic Jurkat cells is depicted as solid line histograms. (B) Heat-map representation of IgG1˜IgG4 reactivity to apoptotic cells for the 50 most reactive serum samples. The shade of gray corresponds to the level of reactivity (MFI) as reported on the scale.

FIG. 6. Correlation between complement activation and IgG subclasses reactivity to apoptotic cells. The deposition of C4d on the surface of apoptotic Jurkat cells was detected by flow cytometry after complement activation in vitro with IgG purified from the 50 most reactive pre-transplant serum samples. C4d deposition is reported together with IgG1 (A) or IgG3 (B) reactivity to apoptotic cells measured in the same samples.

FIG. 7. Titration of purified serum IgG reactivity to apoptotic Jurkat cells. Serum samples from 4 patients were assessed by flow cytometry for their reactivity to apoptotic cells after serial dilution. Results are reported as log₂ values of the MFI.

FIG. 8. Serum IgM and IgG reactivity to apoptotic cells. The serum IgM and IgG reactivity to apoptotic Jurkat cells was measured by flow cytometry in samples collected pre-transplant from kidney transplant recipients as well as healthy subjects. Log₂ values of MFI are reported (y-axis). The numbers of samples tested in each group are shown below the box plot. The horizontal bar represents the median value; the bottom and top of each box represent the 25th and 75th percentiles; the lower and upper bars of each box represent the minimum and maximum values.

FIG. 9. Serum IgM, IgG and purified IgG concentration. (A) Serum IgM, IgG and purified IgG concentrations in pre-transplant serum samples and healthy subjects. The numbers of samples analyzed in each group are shown below the box plot. The horizontal bar represents the median value; the bottom and top of each box represent the 25th and 75th percentiles; the lower and upper bars of each box represent the minimum and maximum values. (B) Correspondence between serum IgG concentration before and after purification. Serum IgG concentrations (x-axis) for all patients (N=300) are plotted with purified IgG concentrations (y-axis) for the same patients. Statistical analysis is based on a two-tailed non parametric spearman's test.

FIG. 10. Comparison of purified serum IgG reactivity to apoptotic cells between patients with autoimmune disease and patients with non-autoimmune disease. Purified IgG reactivity to apoptotic cells (log₂ MFI; y axis) is shown for patients grouped by autoimmune (primary FSGS, IgA nephropathy, Type I diabetes, SLE, Immune complex diseases) or non-autoimmune original diseases. The numbers of samples analyzed in each group are shown below the box plot. The original cause of end-stage renal disease was unknown for eleven patients. The horizontal bar represents the median value; the bottom and top of each box represent the 25th and 75th percentiles; the lower and upper bars of each box represent the minimum and maximum values.

FIG. 11. Purified IgG reactivity to apoptotic wild type and class I negative Jurkat cells. (A) HLA class I expression on wild type and β-2 microglobulin knocked down Jurkat cells. Filled gray histograms show the signal generated by staining with isotype control antibody alone. HLA class I expression on wild type and β-2 microglobulin knocked down Jurkat are depicted as orange and green solid line histograms, respectively. (B) Correspondence between purified IgG reactivity to apoptotic wild type and HLA class I negative Jurkat cells. Purified IgG reactivity to apoptotic wild type and HLA class I negative Jurkat cells were detected in 39 selected patients with high reactivity to HLA class I (MFI>1000). The reactivity to apoptotic wild type Jurkat cells (x-axis) are plotted with reactivity to apoptotic HLA class I negative Jurkat cells (x-axis) for the same patients. Statistical analysis is based on a two-tailed non parametric spearman's test.

FIG. 12. Purified IgG reactivity to viable and apoptotic Jurkat cells. (A) Purified IgG reactivity to viable and apoptotic Jurkat cells are shown for 3 representative pre-transplant patient samples. Results are reported after gating on viable cells (upper panel, blue solid line) or apoptotic cells (lower panel, red solid line). Filled gray histograms show the signal generated by staining with the secondary antibody alone. (B) Pre-transplant purified IgG reactivity to viable Jurkat cells is reported for patients with functioning grafts and patients who experienced graft loss. The numbers of samples analyzed in each group are shown below the scatter plots. Each dot represents a patient. The black bars give the median value for each group. Red dot line represents value (MFI) generated by staining with the secondary antibody alone.

FIG. 13. Concentration of IgG subclasses before and after purification. The concentration of the 4 different IgG subclasses (IgG1˜IgG4) was assessed in 15 randomly picked pre-transplant serum samples patients before (A) and after (B) IgG gel purification. Serum samples were diluted 1:10 during IgG purification. The distribution of all subclasses concentration is depicted with color-coded stacked bars.

FIG. 14. Expression of IgG subclasses on CD19+ B cells from healthy subjects. Peripheral Blood Mononuclear Cells (PBMC) from two healthy subjects were stained using anti-CD19 APC (BD Bioscience) and anti-human IgG1, IgG2, IgG3 and IgG4-PE (Southern Biotech), respectively. Expression of IgG subclasses was assessed after gating on CD19+ cells by flow cytometry.

FIG. 15. Complement activation and C4d deposition. C4d deposition on apoptotic and viable Jurkat cells was assessed by flow cytometry after complement activation by IgG purified from a representative pre-transplant patient serum sample. Results are reported after gating on viable cells (upper panel, blue solid line) or apoptotic cells (lower panel, red solid line). Filled gray histograms show the signal generated by staining with the secondary antibody alone.

In the Summary above, in the Detailed Description, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiment of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

### DEFINITIONS

Unless defined otherwise, technical and scientific terms used herein have the same meaning as commonly understood by one of ordinary skill in the art to which this invention belongs. The practice of the present invention will employ, unless indicated specifically to the contrary, conventional methods of molecular biology and recombinant DNA techniques within the skill of the art, many of which are described below for the purpose of illustration. Such techniques are fully explained in the literature. See, e.g., Singleton et al., Dictionary of Microbiology and Molecular Biology 3rd.sup.ed., J. Wiley & Sons (2001); March, Advanced Organic Chemistry Reactions, Mechanisms and Structure 5th.sup.ed., J. Wiley & Sons (2001); Sambrook & Russell, eds., Molecular Cloning: A Laboratory Manual 3rd ed., Cold Spring Harbor Laboratory Press (2001); Glover, ed., DNA Cloning: A Practical Approach, vol. I & II (2002); Gait, ed., Oligonucleotide Synthesis: A practical approach, Oxford University Press (1984); Herdewijn, ed., Oligonucleotide Synthesis: Methods and Applications, Humana Press (2005); Hames & Higgins, eds., Nucleic Acid Hybridisation: A Practical Approach, IRL Press (1985); Buzdin & Lukyanov, eds., Nucleic Acid Hybridization: Modern Applications, Springer (2007); Hames & Higgins, eds., Transcription and Translation: A Practical Approach, IRL Press (1984); Freshney, ed., Animal Cell Culture, Oxford UP (1986); Freshney, Culture of Animal Cells: A Manual of Basic Technique and Specialized Applications, 6th ed., John Wiley & Sons (2010); Perbal, A Practical Guide to Molecular Cloning, 3rd ed., Wiley-Liss (2014); Farrell, RNA Methodologies: A Laboratory Guide for Isolation and Characterization, 3rd ed., Elsevier/Focal Press (2005); Lilley & Dahlberg, eds., Methods in Enzymology: DNA Structures, Part A: Synthesis and Physical Analysis of DNA, Academic Press (1992); Harlow & Lane, Using Antibodies: A Laboratory Manual: Portable Protocol no. 1, Cold Spring Harbor Laboratory Press (1999); Harlow & Lane, eds., Antibodies: A Laboratory Manual, Cold Spring Harbor Laboratory Press (1988); Seethala & Fernandes, eds., Handbook of Drug Screening, Marcel Dekker (2001); and Roskams & Rodgers, eds., Lab Ref: A Handbook of Recipes, Reagents, and Other Reference Tools for Use at the Bench, Cold Spring Harbor Laboratory (2002) provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. The present invention is in no way limited to the methods and materials described. For convenience, certain terms employed herein in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

“Apoptosis” refers to a natural process of self-destruction by degradative enzymes in certain cells that are genetically programmed to have a limited lifespan or are damaged, as by irradiation or toxic drugs. Also called programmed cell death, a genetically directed process of cell self-destruction that is marked by the fragmentation of nuclear DNA, is activated either by the presence of a stimulus or removal of a suppressing agent or stimulus, and is a normal physiological process eliminating DNA-damaged, superfluous, or unwanted cells. Apoptosis can be induced in vitro using various methods as described below.

An “apoptotic cell” refers to a cell that is dying. In embodiments of the assays described herein, apoptosis is induced in vitro as described herein.

“Biological sample” refers to a sample of blood, plasma, serum or other blood derived products, csf, synovial fluid, bronchioalveolar lavage and ascites.

A “subject” is a mammal, typically a human, but optionally a mammalian animal of veterinary importance, including but not limited to horses, cattle, sheep, dogs, and cats. In some embodiments a “subject” refers to either one who has been received a transplant/graft (post-transplant/graft) or one who is awaiting a transplant (a pre-transplant/graft subject). Subject also includes control or normal subjects who have not had and are not in need of a transplant. pre-transplant and pre-graft subjects. Pre-transplant and pre-graft are used interchangeably herein.

“Organ/tissue transplantation or graft” is the moving of an organ or tissue from one body to another to replace the recipient's damaged or absent organ/tissue. Organs that can be transplanted are the heart, kidneys, liver, lungs, pancreas, intestine, and thymus. Tissues that can be transplanted include transplantation is the moving of a tissue from one body to another or from a donor site to another site in the person's own body. Tissues include bones, tendons (both referred to as musculoskeletal grafts), cornea, skin, heart valves, nerves and veins.

“Transplant/graft rejection” as used herein means the rejection of transplanted organs or tissue by the recipient's immune system, which destroys the transplanted tissue.

“Late transplant/graft rejection” as used herein means rejection that is apparent after approximately 1 year post-transplant.

“At High Risk of Transplant rejection” means that a pre-transplant subject or a subject that has had a transplant will more likely reject the transplant than the average transplant recipient.

“Presensitization or sensitization” as used here means the presence of preformed antibodies in the serum of prospective transplant recipients, particularly alloantibodies against HLA antigens and/or ABO blood group antigens and based on the discoveries made by the inventors, the presence of significantly elevated levels of anti-apoptotic IgG antibodies (against apoptotic cells.)

“Immunosuppressive agent,” “immunosuppressive drug,” and “drug,” are used interchangeably herein, and refer to any agent which inhibits or prevents an immune response against the transplanted tissue following a transplant procedure. Exemplary agents include, but are not limited to, dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triaminolone, anecortave acetate, flurometholone, medrysone, and prednislone.

The term “monitoring” is used herein to describe the use of gene sets to provide useful information about an individual or an individual's health or disease status. “Monitoring” can include, determination of prognosis, risk-stratification, selection of drug therapy, assessment of ongoing drug therapy, prediction of outcomes, determining response to therapy, diagnosis of a disease or disease complication, following progression of a disease or providing any information relating to a patient's health status.

As used herein, the term “diagnosis” includes the detection, typing, monitoring, dosing, and comparison, at various stages of prostate cancer in a subject. Diagnosis includes the assessment of a predisposition or risk of rejecting a transplant in the future, which is useful to define the most appropriate treatment.

As used herein, the terms “treat,” “treatment,” “treating,” or “amelioration” refer to therapeutic treatments for transplant rejection known in the art, wherein the object is to reverse, alleviate, ameliorate, inhibit, slow down or stop the progression of transplant rejection, or reduce the severity of a symptom or condition associated with transplant rejection. The term “treating” includes reducing or alleviating at least one adverse effect or symptom of a condition. Treatment is generally “effective” if one or more symptoms or clinical markers of transplant rejection are reduced.

By “contacting” is meant an instance of exposure of the extracellular surface of an apoptotic cell to IgG or other substance at physiologically effective levels. An apoptotic cell can be contacted with IgG antibodies by adding the IgG antibodies to the culture medium. The duration of “contact” of the apoptotic cell(s) with the IgG antibodies is determined by the time the IgG antibodies are present in the medium bathing the apoptotic cell(s). The contacting step in the methods of the present invention takes place in vitro.

The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity. The terms “antibody” and “antibodies” broadly encompass naturally-occurring forms of antibodies (e.g., IgG, IgA, IgM, IgE.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

The term “IgG antibody” as used herein means a class of immunoglobulins including the most common antibodies circulating in the blood that facilitate the phagocytic destruction of microorganisms foreign to the body, that bind to and activate complement, and that are the only immunoglobulins to cross over the placenta from mother to fetus. Immunoglobulin G (IgG) is an antibody isotype. It is a protein complex composed of four peptide chains—two identical heavy chains and two identical light chains arranged in a Y-shape typical of antibody monomers. Each IgG has two antigen binding sites. Representing approximately 75% of serum immunoglobulins in humans, IgG is the most abundant antibody isotype found in the circulation. IgG molecules are synthesized and secreted by plasma B cells. There are four IgG subclasses (IgG 1, 2, 3, and 4) in humans, named in order of their abundance in serum (IgG1 being the most abundant).

| | | | Binds to Fc receptor |
|---|---|---|---|
| Name | Percent | Complement activator | on phagocytic cells |
| IgG1 | 66% | second-highest | high affinity |
| IgG2 | 23% | third-highest | extremely low affinity |
| IgG3 | 7% | highest | high affinity |
| IgG4 | 4% | no | intermediate affinity |

“Anti-apoptotic cell IgG” as used here means IgG that binds to apoptotic cells in embodiments of the present methods.

“Control level” and “normal level of expression” as used in the embodiments of the present methods refer to a median value of serum IgG in biological samples from at least 3 controls, normal healthy subjects that have not had and are not in need of a transplant.

“Threshold” or “threshold level” as used herein refers to a level or range of levels that separate normal level of expression of IgG antibodies in a control population from a level or range of levels of expression of IgG antibodies in a pre-transplant subject or a subject that has had a transplant, wherein a high risk of transplant rejection within at least one year is diagnosed if the threshold is reached or exceeded. In certain of the present embodiments the threshold is a value that is at least equal to the median value of at least three control subjects+2 standard deviations.

### DETAILED DESCRIPTION

In the following description, for the purposes of explanation, numerous specific details are set forth in order to provide a thorough understanding of the present invention. It will be apparent, however, to one skilled in the art that the present invention may be practiced without these specific details.

Presensitization of pre-transplant and pre-graft subjects has been a long-standing limitation to solid organ transplantation that has been explained by the presence of allospecific antibodies reactive to donor HLA or ABO blood group antigens. It has now been discovered that significantly elevated levels of anti-apoptotic cell IgG is an important contributor to and predictor of late graft rejection. Kaplan-Meier survival analysis revealed that patients with high pre-transplant IgG reactivity to apoptotic cells had a significantly increased rate of late graft loss that was apparent after approximately 1 year post-transplant. Importantly, the association between pre-transplant IgG reactivity to apoptotic cells and graft loss was still significant after excluding patients with high reactivity to HLA. This reactivity was almost exclusively mediated by IgG1 and IgG3 with complement fixing and activating properties. The association between elevated levels of anti-apoptotic cell IgG antibodies and late transplant rejection was seen in both pre- and post-transplant subjects and suggests a direct physiological role in graft destruction.

Although the experiments and data used in the studies reported here are derived from kidney transplant patients, the underlying principles apply to any pre- or post-transplant subject. The present invention provides a method for use in clinical practice to prospectively identify pre-transplant and post-transplant patients who are at high risk of progression to transplant rejection. The prospective identification of patients at high risk for transplant rejection enables rational institution of interventional therapy to reduce anti-donor allo-reactivity that either delays or prevents transplant/graft rejection. Based on the results herein described, certain embodiments of the invention are directed to methods for identifying pre- and post-transplant subjects who are at a high risk of late transplant rejection (after one year) by determining that these subjects have significantly elevated levels of anti-apoptotic cell IgG compared to control, normal subjects. An elevated level is defined a value above or equal to the median value of at least three control subjects+2 standard deviations. Other embodiments include treating pre- or post-transplant subjects at a high risk of transplant/graft rejection aggressively with immunosuppression or other desensitization protocols to reduce the risk of, or eliminate or delay transplant rejection.

The present invention enables identification of subjects who are at risk for high-grade rejection of a transplant, thereby reducing unnecessary diagnostic and therapeutic procedures in low risk patients. In a cardiac transplant, for example, identifying high risk individuals has the potential to significantly reduce the number of endomyocardial biopsies being performed during the first year, since these procedures would not be required or would be done less frequently in patients at low risk of rejection. In addition, the present invention enables the identification of low risk patients in whom immunosuppressive reagents may be safely withdrawn or reduced, thereby eliminating potential side effects. Following withdrawal or reduction of immunosuppression, the methods of the invention may be further utilized by the attending physician to monitor changes in the transplant recipient's risk of rejection. Such monitoring can be performed, for example, at intervals of every three months. Should the recipient's risk shift from low risk to high risk the physician may choose to resume treatment with an immunosuppressive agent.

In order that the invention may be readily understood and put into practical effect, particular preferred embodiments will now be described by way of the following non-limiting examples.

### Overview

Pre-sensitization has been a major limitation to solid organ transplantation for decades and candidate recipients with pre-existing antibodies to potential donor grafts have a higher risk of rejection and eventually graft loss. It is commonly accepted that these antibodies are either naturally pre-formed or had developed after exposure to allogeneic antigens occurring during pregnancy, blood transfusion or previous allografts. In ABO compatible donor-recipient pairs, sensitizing antibodies are primarily IgG reactive to human leukocyte antigen (HLA). However, a number of observations suggest that non-HLA reactive antibodies also contribute to pre-sensitization and may influence the overall graft outcome. Cases of early humoral rejection in the absence of detectable donor-specific antibodies (DSA) have also been reported.

In a landmark collaborative transplant study, Opelz and colleagues revealed the association between high panel reactive antibodies (PRA) before transplantation and late graft loss in recipients of kidney transplants from HLA that could not be attributed to donor specific HLA antibodies. Additional studies support a contribution of non-HLA antibodies to pre-sensitization. More specifically, serum IgG reactivity to autoantigens such as cardiac myosin, vimentin, collagen, oxidized lipids and LG3 has been associated with increased rejection rates and reduced graft survival.

In previous studies, a number of B cell clones secreting antibodies reactive to apoptotic cells were isolated from a kidney transplant recipient with antibody mediated rejection (AMR). Elevated IgG reactivity to apoptotic cells in kidney transplant recipients experiencing AMR was observed compared to patients with stable graft function. Collectively, these findings alluded to a contribution of anti-apoptotic cell IgG to the pathophysiology of graft rejection, however, none of these studies examined the contribution of anti-apoptotic cell IgG to pre-sensitization of transplant subjects or to the risk of rejection in post-transplant subjects.

### Results

1. Pre-transplant serum IgG reactivity to apoptotic cells was significantly higher in serum from 300 kidney transplant recipients compared to 20 healthy controls using either purified or non-purified IgG. Binding of purified IgG to apoptotic class I negative cells was comparable to that of wild type apoptotic Jurkat cells in the 39 patients with high IgG reactivity to HLA class I, indicating that these IgG antibodies recognize other antigenic structures than HLA on apoptotic cells. However, no reactivity was detected on viable Jurkat cells.

2. Forty-six patients lost their transplants/grafts and returned to dialysis due to various complications. The mean duration of follow-up for all patients included in this retrospective study was 81.2±35.3 months. The patients that suffered transplant rejection had significantly higher purified IgG reactivity to apoptotic cells before transplantation compared to those transplant patients who did not reject the transplant/graft (P<0.001). Elevated pre-transplant IgG reactivity to apoptotic cells was shown to be an explanatory factor that is significantly associated with eventual transplant/graft loss even when the reactivity to HLA class I, class II and MICA ((P=0.003, P=0.003, P=0.023, respectively) as well as other variables were included and adjusted for in the statistical model.

3. For all specimens the purified serum IgG reactivity to apoptotic cells was almost exclusively mediated by complement fixing IgG1 or IgG3 subclasses or a combination of the two, and tests further showed that these antibodies retained the capacity to activate complement.

### EMBODIMENTS

Based on the results, certain embodiments are directed to methods for predicting transplant rejection in a pre-transplant subject or a subject who has had a transplant, typically a mammal, more preferably a human, by (a) obtaining a biological sample from the pre- or post-transplant subject and a biological sample from each of at least 3 healthy subjects; (b) isolating IgG antibodies from the pre- or post-transplant sample and from the at least three healthy subject samples; (c) contacting a test population of apoptotic cells with the pre- or post-transplant subject IgG antibodies and contacting at least three control populations of apoptotic cells with the IgG antibodies from each of the control samples, for a time and under conditions that permit the antibodies to bind to the apoptotic cells; and (d) determining the amount of binding of the IgG antibodies to apoptotic cells in the test population and in the control populations, and if the amount of IgG antibody binding to apoptotic cells in the test population is higher than the median amount of IgG antibody binding to apoptotic cells in the three control populations+two standard deviations, then determining that the subject is at a high risk of transplant rejection. In other embodiments the apoptotic cells used in the assay do not express HLA class 1 antigens. Any apoptotic cell can be used in the embodiments including Jurkat cells, 293 Human Embryonic Kidney cells, and endothelial cells including human umbilical cord endothelial cells.

The IgG antibodies are isolated from the biological sample and typically undergo at least one purification step as described. Any method known in the art for isolating and purifying antibodies from blood products can be used, including also separation using protein G and ammonium sulfate precipitation.

In some embodiments, the apoptotic cells are treated with UV light to induce apoptosis or they become apoptotic by activating the Fas receptor with agonist antibody, or administering doxorubicin, 5-fljorouracil, paclitaxel, vinblastine, camptothecin or staurosporine. Many different biological and chemical methods for inducing apoptosis, and for assaying the induction of this cellular process, are routinely performed including ultraviolet B irradiation, small molecule drug treatments, death receptor ligation, and exposure to granule components of cytotoxic lymphocytes. Assays are available to confirm the induction of apoptosis by quantifying changes in mitochondrial membrane potential, phosphatidylserine membrane localization and fragmented DNA content.

In some embodiments, a desensitization treatment is administered if it is determined that the subject is at high risk of transplantation rejection. Desensitization can be achieved by administering a therapeutically effective amount of an immunosuppressant drug selected from the group consisting of Bortezomib, cyclosporine, rapamycin, Campath I, thymoglobulin, (rATG), anti-thymocytic antibody, Rituximab, and Gamimune N, intravenous immune Globulin (IVIG). In some embodiments unwanted antibodies, including anti-HLA and blood group alloantibodies are removed with plasmapheresis.

Other embodiments are directed to kits for use in the assay for predicting transplant/graft rejection. A kit can include a biological sample collection device to obtain a serum sample from a subject; and any one or combinations of: serum samples from a healthy control subject or more than one healthy subject comprising IgG antibodies to be incubated with the apoptotic cells, apoptotic cells for the assay, anti-IgG secondary antibodies optionally labeled to facilitate detection, for example using flow cytometry with fluorescently labeled secondary antibodies or peroxidase. Other labels include a radioactive label, a fluorophor, a phosphor, a laser dye, a chromogenic dye, a macromolecular colloidal particle, a latex bead which is colored, magnetic or paramagnetic, an enzyme which catalyzes a reaction producing a detectable result or the label is a tag. An immunosorbent assay to detect anti-apoptotic antibodies is being developed, including for example, an antibody directed to IgG that is conjugated to a label or enzyme, flow cytometry, or ELISA.

In some embodiments elevated anti-apoptotic IgG antibody levels post-transplant are evaluated along with post-transplant levels of other markers of rejection including anti-HLA Class I/II antibodies, serum creatinine and proteinuria to predict a higher risk of transplant rejection.

### Methods of Making Apoptotic Cells

In some embodiments, the apoptotic cells are treated with UV light to induce apoptosis or they become apoptotic by activating Fas (such as by exposure to an anti-FAS antibody) or TNF-receptors, by crosslinking the receptors with agonist antibody, or administering doxorubicin, 5-fljorouracil, paclitaxel, vinblastine, camptothecin or staurosporine. Staurosporine, isolated from Streptomyces, is a protein kinase inhibitor. Staurosporine induces DNA fragmentation and apoptosis in Jurkat cells at 1 mM in 2-3 hours. Camptothecin, isolated from the Chinese herb xi shu (Camptotheca acuminata), is an inhibitor of DNA topoisomerase I. Camptothecin induces apoptosis at 4 mg/mL in Jurkat cells at S-phase of the cell cycle. Tumor Necrosis Factor-alpha (TNF-α) is a human recombinant protein expressed in E. coli as a single, non-glycosylated, polypeptide of 158 amino acids. Tumor Necrosis Factor-beta (TNF-β): human recombinant protein expressed in E. coli as a single, non-glycosylated, polypeptide of 171 amino acids. Many of these reagents are available from ImmunoChemistry Technologies LLC. There is also a growing body of evidence indicating that nitric oxide is able to induce apoptosis by helping to dissipate the membrane potential of mitochondria and therefore make it more permeable.

A cell undergoing apoptosis shows a characteristic morphology:
1. Cell shrinkage and rounding are shown because of the breakdown of the proteinaceous cytoskeleton by caspases.
2. The cytoplasm appears dense, and the organelles appear tightly packed.
3. Chromatin undergoes condensation into compact patches against the nuclear envelope (also known as the perinuclear envelope) in a process known as pyknosis, a hallmark of apoptosis.
4. The nuclear envelope becomes discontinuous and the DNA inside it is fragmented in a process referred to as karyorrhexis. The nucleus breaks into several discrete chromatin bodies or nucleosomal units due to the degradation of DNA.
5. The cell membrane shows irregular buds known as blebs.
6. The cell breaks apart into several vesicles called apoptotic bodies, which are then phagocytosed.

Apoptosis may be induced in experimental systems through a variety of methods. In general, they can be divided into 2 categories: a) biological induction; and b) chemical induction.

### A) Biological Induction of Apoptosis

Activation of either Fas or TNF-receptors by the respective ligands or by crosslinking with agonist antibody induces apoptosis of Fas- or TNF receptor-bearing cells. Below is an example general protocol used to induce apoptosis using anti-Fas mAb in Jurkat Cells. The specific protocol used in the experiments described herein is set forth in the Examples.
1. Grow Jurkat cells in RPMI-1640 medium containing 10% fetal bovine serum in a humidified, 5% CO2 incubator at 37° C.
2. Suspend the cells in fresh medium at a concentration of 1×105 cells/ml. After two to three days of incubation in a 37° C., 5% CO2 incubator, harvest the cells by centrifugation at 300-350×g for 5 mins.
3. Resuspend cells in fresh medium to 5×105 cells/ml and add anti-Fas mAb to a final concentration of 0.05-0.1 μg/ml.
4. Incubate for 3-6 hours in a 37° C. incubator. As a negative control, incubate untreated cells (no anti-Fas mAb) under the same conditions. (Stop here for homogeneous assay, or plate the cells in a 96-well plate.)
5. Harvest the cells by centrifugation at 300-350×g for 5 mins.
6. Remove all medium and resuspend cells in PBS.
7. Repeat centrifugation and resuspend the cell pellet in PBS to 1.5×106 cells/ml.
8. Proceed to apoptosis detection.

### Chemical Induction of Apoptosis

Depending on the agent selected and the concentrations used, maximal induction of a particular protein may occur within 8 to 72 hours post-treatment. However, not all proteins are affected by reagents in a particular cell line. The following protocol is based on p53-dependent G1-arrest that occurs in response to DNA damage by chemical agents. A typical time course for p53 induction is 40 to 48 hours treatment with a DNA damaging agent. A sample protocol follows:
1. Inoculate each of 2 or more 10 cm2 tissue culture dishes for adherent cells or T-75 flasks for non-adherent cells with approximately 1×106 cells. One dish or flask will be used as negative control for non-induced or basal level expression.
2. Confirm that cells are growing by visual inspection of tissue culture dishes or by viable cell counts on non-adherent cells in T-75 flasks. Add DNA damaging agents to recommended final concentrations. The list below gives suggestions of final concentrations that can be used for several well-known apoptosis inducing chemicals:

### EXAMPLES

0.2 μg/ml Doxorubicin (stock prepared in H2O, 25 μg/ml)

5-Fluorouracil (stock prepared in DMSO)

100-58 nM Paclitaxel (stock prepared in DMSO)

60 nM Vinblastine (stock prepared in methanol)

1 mM staurosporine in DMSO. Add appropriate volume of buffer or solvent (i.e. DMSO) to the non-induced control.
3. Check cells to determine if cells have begun to apoptose. This can be assessed by checking the morphology of the cells (cells will become granulated and blebbing may be observed). Viability can be checked using e.g. trypan blue cell counting. Harvest cells if greater than 75% of the cells appear to have died upon trypan blue viability counting.
4. Harvest cells and prepare lysates for either western blotting or immunoprecipitation. For any agent used, a time course of induction can be performed by inoculating additional dishes or flasks and harvesting at various times (i.e. 24, 48 and 72 hours) after addition of the DNA damaging agent. For the examination of apoptotic proteins, dead cells should also be collected. Always compare levels of p53 from treated cells and controls to confirm induction.

Other methods for inducing apoptosis, and for assaying the induction of this cellular process, are routinely performed using death receptor ligation, and exposure to granule components of cytotoxic lymphocytes. Assays also available to confirm the induction of apoptosis by quantifying changes in mitochondrial membrane potential, phosphatidylserine membrane localization, DNA content, and autoantigen.

### Treatment Options for Patients Identified as High Risk for Transplant Rejection

Panel Reactive Antibody (PRA) is an immunological laboratory test routinely performed on the blood of people awaiting organ transplantation. The PRA score is expressed as a percentage between 0% and 99%, and it represents the proportion of the population to which the person being tested will react via pre-existing antibodies. These antibodies target the Human Leukocyte Antigen (HLA), a protein found on most cells of the body. Each population will have a different demographic of HLA antigens, and so the PRA test will differ from country to country. A high PRA score usually means that the individual is primed to react immunologically against a large proportion of the population. Individuals with a high PRA are often termed “sensitized”, which indicates that they have been exposed to “foreign” (or “non-self”) proteins in the past and have developed antibodies to them. These antibodies develop following previous transplants, blood transfusions and pregnancy. Transplanting organs into recipients who are “sensitized” to the organs significantly increases the risk of rejection, resulting in higher immunosuppressant requirement and shorter transplant survival. People with high PRA score therefore spend longer waiting for an organ to which they have no pre-existing antibodies.

Most of the current protocols to prevent or reduce the risk of transplant rejection involve immunosuppression that is deliberately induced typically with drugs but it may also involve surgery (spleen removal), plasmapharesis, or radiation. The discovery of cyclosporine in 1970 that allowed for significant expansion of kidney transplantation to less well-matched donor-recipient pairs as well as broad application of liver transplantation, lung transplantation, pancreas transplantation, and heart transplantation. Immunosuppressive drugs can be classified into different groups. Glucocorticoids inhibit various inflammatory events: epithelial adhesion, emigration, chemotaxis, phagocytosis, respiratory burst, and the release of various inflammatory mediators (lysosomal enzymes, cytokines, tissue plasminogen activator, chemokines, etc.) from neutrophils, macrophages, and mastocytes. Cytostatics inhibit cell division and for immunotherapy, they are used in smaller doses than in the treatment of malignant diseases. They affect the proliferation of both T cells and B cells. Due to their highest effectiveness, purine analogs are most frequently administered. Azathioprine is the main immunosuppressive cytotoxic substance extensively used to control transplant rejection reactions. It is non-enzymatically cleaved to mercaptopurine that acts as a purine analogue and an inhibitor of DNA synthesis. Mercaptopurine itself can also be administered directly. Among these, dactinomycin is the most important. Cytotoxic antibiotics are also used including anthracyclines, mitomycin C, bleomycin, and mithramycin.

Antibodies are sometimes used as a quick and potent immunosuppressive therapy to prevent the acute rejection reactions. polyclonal antibodies inhibit cell-mediated immune reactions, including graft rejection, however, because of a high immunogenicity of polyclonal antibodies, adverse side effects include an acute reaction to the treatment. It is characterized by fever, rigor episodes, and even anaphylaxis. Monoclonal antibodies have fewer side effects because they are directed towards exactly defined antigens. Especially significant are the IL-2 receptor- (CD25-) and CD3-directed antibodies.

Muromonab-CD3 (trade name Orthoclone OKT3, marketed by Janssen-Cilag) is an immunosuppressant drug given to reduce acute rejection in patients with organ transplants.[1][2] It is a monoclonal antibody targeted at the CD3 receptor, a membrane protein on the surface of T cells. It was the first monoclonal antibody to be approved for clinical use in humans.[2] drugs acting on immunophilins, however this drug has fallen out of favor because it can cause excessive immunosuppression. Two chimeric mouse/human anti-Tac antibodies in the year 1998: basiliximab (Simulect) and daclizumab (Zenapax). These drugs act by binding the IL-2a receptor's α chain, preventing the IL-2 induced clonal expansion of activated lymphocytes and shortening their survival. They are used in the prophylaxis of the acute organ rejection after bilateral kidney transplantation, both being similarly effective and with only few side-effects.

Rituximab (Anti-CD20) is a chimeric murine/human monoclonal antibody that binds to CD20 on pre-B and mature B lymphocytes. It is FDA approved for treatment of refractory or relapsed B cell lymphomas and is also used for treatment of post-transplant lymphoproliferative disease (PTLD)Rituximab has been used off label in de-sensitization protocols for incompatible kidney transplantation (ABO-incompatible or cross-match positive) or in the treatment of AMR as a single dose of 375 mg/m².

Bortezomib (a proteosomal Inhibitor) is a tri-peptide and proteosomal inhibitor approved by the FDA for the treatment of multiple myeloma in which it was shown to cause apoptosis of normal plasma cells, thereby having the potential to decrease alloantibody production in sensitized patients.

Complement inhibitors have also been used to treat or prevent transplant rejection. Eculizumab is a humanized monoclonal antibody against complement protein C5 that binds to C5 protein with high affinity, thereby inhibiting its cleavage to C5a and C5b and preventing generation of the terminal complement complex C5b-9. This process halts complement-mediated cell destruction. The FDA has approved eculizumab for the treatment of paroxysmal nocturnal hemoglobinuria and it has been used in the prevention and treatment of atypical hemolytic-uremic syndrome after transplantation. This treatment may be particularly effective because the antiapoptotic IgG antibodies are IgG1 and 3 which involve complement.

Tacrolimus (trade name Prograf) is a product of the bacterium Streptomyces tsukubaensis. It is a macrolide lactone and acts by inhibiting calcineurin. Like tacrolimus, ciclosporin (Novartis' Sandimmune) is a calcineurin inhibitor (CNI). It has been in use since 1983 and is one of the most widely used immunosuppressive drugs. It is a cyclic fungal peptide, composed of 11 amino acids. Sirolimus (rapamycin, trade name Rapamune) is a macrolide lactone, produced by the actinomycete bacterium Streptomyces hygroscopicus. It is used to prevent rejection reactions. Although it is a structural analogue of tacrolimus, it acts somewhat differently and has different side-effects. Contrary to ciclosporin and tacrolimus, drugs that affect the first phase of T lymphocyte activation, sirolimus affects the second phase, namely signal transduction and lymphocyte clonal proliferation. Interferons, opioids, mycophenolate and TNF inhibitors can also be used for immunosuppression.

Other treatment for desensitization and transplant rejection include IVIG immunoglobulin treatment (IVIG) or plasmapheresis (PP) with low-dose IVIG (Akalin E: Posttransplant immunosuppression in highly sensitized patients. Contrib Nephrol 162: 27-34, 2009). IVIG has been FDA approved for allogeneic bone marrow transplant and kidney transplant. Some protocols for desensitization are summarized below in Table 1.

Treatment options for sensitized patients

```
1. Removal of antibodies by PP or IA
2. Inhibition of antibody production
    a. Anti-B cell agents: rituximab (anti-CD20)
    b. Plasma cell inhibitors: bortezemib (proteosome inhibitor)
3. Inhibition of complement cascade: eculizumab (anti-C5a)
4. IVIG has multiple effects on different immune pathways:
    a. Neutralization of circulating anti-HLA antibodies through anti-idiotypic antibodies
    b. Inhibition of complement activation by binding C3b and C4b and neutralization of C3a
and C5a
    c. Blockage of immune activation and enhancing the clearance of anti-HLA antibodies by
competing for activating FcyRs
    d. Inhibits the expression CD19 on activated B cells and induces apoptosis of B cells
    e. Induces the expression of FcyIIB, which is a negative regulatory receptor on immune
  cells
PP, Plasmapheresis; IA, immunoadsorption; IVIG, intravenous Ig.
```

Plasmaperesis (PP) and immunoadsorption (IA) techniques have been used to remove alloantibodies. PP is not specific for Ig removal and results in a lowering of all plasma proteins, including clotting factors, and requires replacement with fresh frozen plasma and albumin. IA includes a sepharose-bound staphylococcal protein A column with a high affinity for binding IgG and developed to remove IgG antibodies. The advantages of IA over PP include specificity, a greater amount of antibody removal, and the elimination of the need to replace large volumes of plasma. One 3- to 4-hour treatment course with IA results in a 15% to 20% reduction and three to six courses of treatment result in >90% reduction in plasma IgG levels. However, anti-HLA antibody titers rebound and return to baseline levels within a few weeks after the completion of PP or IA (Hakim R M, Milford E, Himmelfarb J, Wingard R, Lazarus J M, Watt R M: Extracorporeal removal of anti-HLA antibodies in transplant candidates. Am J Kidney Dis 16: 423-431, 1990). Most columns available in Europe and Japan for IA are not approved by the U.S. Food and Drug Administration (FDA) for clinical use in the United States.

Intravenous Ig (IVIG) therapy is used to treat sensitized patients. IVIG is a blood product administered intravenously. It contains the pooled, polyvalent, IgG antibodies extracted from the plasma of over one thousand blood donors. IVIG's effects last between 2 weeks and 3 months. Various mechanisms have been proposed to explain how IVIG works, including by neutralizing circulating anti-HLA antibodies through anti-idiotypic antibodies, by inhibiting complement activation but not anti-idiotypic activity, by preventing the generation of the C5b-C9 membrane-attack complex or by blocking immune activation and enhancing the clearance of anti-HLA antibodies. IVIG also has inhibitory effects on cellular immune responses; nonspecific inhibitory effects on the immune system by binding to Fcy receptors on macrophages, neutrophils, platelets, mast cells, and natural killer cells; and inhibits cytokine, chemokine, adhesion molecule, and endothelial cell activity. IVIG has been used in highly sensitized patients at the top of the waiting list in order to decrease PRA levels, in desensitization protocols of ABO-incompatible and cross-match-positive patients, and in the treatment of AMR. The dose of WIG varies among protocols from about 100 mg/kg to 2.0 g/kg and is usually given during a hemodialysis session or as a slow infusion in nondialysis patients.

Some protocols include a short course of high-dose corticosteroids can be applied. Triple therapy adds a calcineurin inhibitor and an anti-proliferative agent. Where calcineurin inhibitors or steroids are contraindicated, mTOR inhibitors are used. Corticosteroids include prednisone and hydrocortisone. Calcineurin inhibitors include cyclosporin. Corticosteroids include prednisone and hydrocortisone. Calcineurin inhibitors include cyclosporin and tacrolimus. Antiproliferatives include azathioprine and mycophenolic acid, and mTOR inhibitors include sirolimus and everolimus.

### EXAMPLES

The invention is illustrated herein by the experiments described by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Example 1

### Materials and Methods

### Patient Characteristics and Sample Collection

The collection of all specimens used in this study was approved by MGH internal review board. The patient group consisted of 300 non-consecutive kidney transplant recipients who received a kidney transplant at MGH between May 1999 and July 2007 and whose pre-transplant serum specimens were available. Patients with pre-transplant DSA were excluded. All serum specimens were collected prior to transplantation as part of the patients' standard clinical care. Serum samples collected from 20 healthy subjects were used as control. The baseline characteristics of all patients included in this study are summarized as Table 1.

TABLE 1
Patient characteristics

| Parameters | Values | |
|---|---|---|
| Age in year (mean ± SD) | 49.0 ± 14.4 | |
| Sampling time pre-tx, month (mean ± SD) | 3.7 ± 2.5 | |
| Follow-up, month (mean ± SD) | 81.2 ± 35.3 | |
| Gender, n (%) | | |
| Male | 192 | (64.0%) |
| Female | 108 | (36.0%) |
| Race, n (%) | | |
| Caucasian | 239 | (79.7%) |
| African | 29 | (9.6%) |
| Asian | 14 | (4.7%) |
| Hispanic | 18 | (6.0%) |
| No. of transplant, n (%) | | |
| First | 259 | (86.3%) |
| Second | 35 | (11.7%) |
| Third | 6 | (2.0%) |
| Donation, n (%) | | |
| Deceased | 147 | (49.0%) |
| Living related | 84 | (28.0%) |
| Living unrelated | 69 | (23.0%) |
| Transfusion pre-Tx, (n %) | | |
| Yes | 94 | (31.3%) |
| No | 206 | (68.7%) |
| Induction therapy, (n %) | | |
| Yes | 84 | (27.9%) |
| No | 216 | (72.1%) |
| Cause of end-stage renal disease, n (%) | | |
| Polycystic kidney | 36 | (12.0%) |
| Focal Segmental Glomerulosclerosis (Primary) | 26 | (8.7%) |
| IgA nephropathy | 39 | (13.0%) |
| Type I diabetes mellitus | 28 | (9.3%) |
| Hypertension | 21 | (7.0%) |
| Obstructive/reflux uropathy/anatomical issues | 22 | (7.3%) |
| Systemic lupus erythematosus | 6 | (2.0%) |
| Immune complex disease | 29 | (9.7%) |
| Congenital/hereditary disease | 18 | (6.0%) |
| Interstitial nephritis | 23 | (7.7%) |
| Focal Segmental Glomerulosclerosis (Secondary) | 12 | (4.0%) |
| Type II diabetes mellitus | 25 | (8.3%) |
| Others | 4 | (1.3%) |
| Unknown | 11 | (3.7%) |

Forty six of the 300 patients lost their grafts and returned to dialysis. For 42 of these patients, the cause of graft loss was based on pathological changes seen in biopsy specimens. For the remaining 4 patients, the cause of graft loss was determined by clinical criteria and serological tests.

### Serum IgG Purification

Serum IgG were purified from patients specimens using the Melon Gel IgG Purification Kit (Thermo Scientific, Rockford, Ill.) according to the manufacturer's instructions. Briefly, serum samples were diluted 1:10 in a dilution buffer and incubated with a resin that retains non-IgG immunoglobulin as well as other abundant serum proteins.

### Assessment of Reactivity to Apoptotic Cells

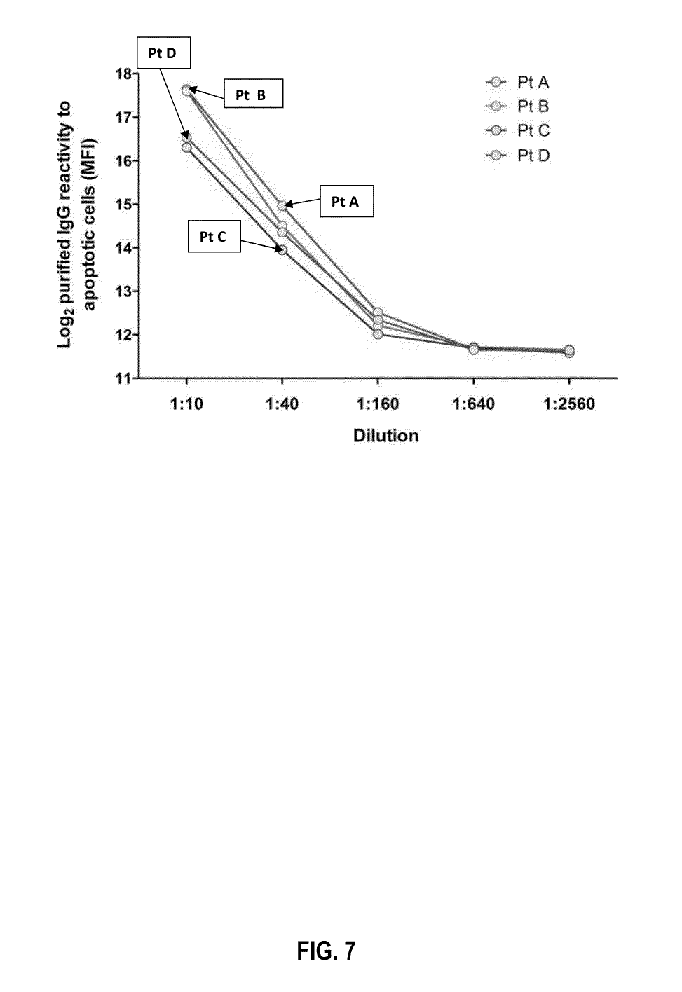

Flow cytometry was used to assess the reactivity of serum IgM, IgG and purified IgG to apoptotic Jurkat cells in samples collected pre-transplant from 300 patients who received a kidney transplant at MGH between 1999 and 2007 as well as 20 control healthy subjects. Human Jurkat T cell leukemia cells were cultured overnight with 200 ng/ml of anti-FAS antibody (clone CH11, Millipore, Billerica, Mass.) or exposed to UV light (240×10⁻³ J) to induce apoptosis using a UV stratalinker 2400 (Stratagene, Santa Clara, Calif.). Then, 1×10⁶ apoptotic Jurkat T cells were incubated for 30 minutes at 37° C. with 100 μl serum IgM and IgG samples diluted 1:5 in PBS or 60 μl purified IgG samples diluted 1:2 in PBS. After two washes in 3 ml PBS at 4° C., samples were incubated with FITC-conjugated anti-IgM or anti-IgG F(ab′)2 secondary antibodies, respectively (Invitrogen, Camarillo, Calif.) for 30 minutes at 4° C. After two additional washes in 3 ml PBS at 4° C., cells were acquired using an Accuri C6 flow cytometer (BD Biosciences, San Jose, Calif.) after gating on apoptotic cells. To avoid inter-experiment variations, all samples were assessed at the same time in the same experiment using the same instrument settings. Results were reported as log₂ values of the mean fluorescence intensity (MFI) of positive cells. A titration experiments carried out with purified IgG samples from 4 patients with highest IgG reactivity to apoptotic cells is reported in FIG. 7.

### Generation of HLA Class I Negative Jurkat Cells

Supernatants containing packaged pLKO.1 lentiviral vector containing a shRNA specific for the human β2-microglobulin was obtained from Dr. Roberto Bellucci (Dana-Farber Cancer Institute). Preparation of the lentiviral vector is described elsewhere (31). Jurkat cells were transduced with virus supernatants and polybrene at 8 μg/ml (Millipore) two times and selected with puromycin for 24 hours after the second transduction. HLA class I negative cells were then purified from transduced cells by sorting. This operation was repeated three times until homogeneous HLA class I negative was obtained.

### Quantification of IgM and IgG Concentration

Serum IgM and IgG in patients and healthy subjects were quantified using a Cytometric Bead Array kit (BD Biosciences, San Jose, Calif.). Briefly, capture beads were incubated with 50 μl samples diluted at 1:2,500 for IgM, 1:100,000 for IgG and 1:10,000 for purified IgG with dilution buffer for 1 h at room temperature. After 1 wash in 1 ml wash buffer, 50 μl mixed PE detection reagent was added in each sample and incubated for 2 h at room temperature. After 1 wash in 1 ml wash buffer, beads were resuspended in 300 μl wash buffer and acquired using an Accuri C6 flow cytometer (BD Biosciences). Immunoglobulin concentrations were calculated using FCAP Array v3.0 software (BD Biosciences). IgG subclasses (IgG1˜IgG4) in patients before and after IgG purification were quantified by ELISA using Human IgG Subclass Profile Kit (Invitrogen, Camarillo, Calif.) according to the manufacturer's instruction. Briefly, 50 μl serum samples diluted at 1:2500 were incubated for 30 minutes at room temperature with anti-human IgG1˜IgG4 subclasses specific antibodies, respectively. Plates were washed 4 times in wash buffer, peroxidase anti-human IgG solution was added and incubated for 30 minutes at room temperature, after 4 washes, developed using 3,3′,5,5′-tetramethylbenzidine. Optical density was measured at 450 nm.

### Detection of Reactivity to HLA and MICA Molecules

The reactivity of patients' serum to HLA Class I, HLA Class II, or MICA was assessed using beads coated with mixed HLA molecules (LABScreen Mixed, One Lambda, Los Angeles, Calif.). Antibodies reactive to beads were detected with an anti-IgG (One Lambda) PE-conjugated secondary antibody on a Luminex 200 apparatus (Luminex, Austin, Tex.). A MFI of 1,000 was arbitrarily used as a cutoff value.

### Serum IgG Subclass Reactivity to Apoptotic Cells

Apoptotic Jurkat (1×10⁶ cells) cells were incubated for 30 minutes at 37° C. with 60 μl IgG purified from the 50 most reactive patient serum specimens diluted 1:2. After two washes in 3 ml PBS at 4° C., samples were incubated with PE-conjugated anti-IgG1, IgG2, IgG3, IgG4 secondary antibodies for each patient, respectively (Clone 4E3, HP6002, HP6050, HP6025, Southern Biotech, Birmingham, Ala.) at 4° C. for 30 minutes. After two washes, cells were acquired using an Accuri C6 flow cytometer (BD Biosciences, San Jose, Calif.) after gating on apoptotic cells.

### C4d Binding Assay

Apoptotic Jurkat cells (0.5×10⁶ cells) were incubated for 30 minutes at 37° C. with 60 μl purified serum IgG (see above) diluted 1:2. Human serum from a healthy subject diluted 1:100 in HBSS was then added as a source of complement and incubated for 15 minutes at 37° C. After 2 washes in PBS, cells were incubated for 30 minutes at 4° C. with an anti-C4d antibody (Quidel, San Diego, Calif.), washed twice in PBS, and then incubated for 30 minutes at 4° C. with a FITC-conjugated anti-mouse IgG secondary antibody (BD Biosciences). After 2 final washes at 4° C., C4d binding was measured after gating on apoptotic cells on an Accuri C6 flow cytometer (BD Biosciences).

### Statistical Analysis

Mann-Whitney tests were performed to compare IgM or IgG reactivity to apoptotic cells between all subgroups of patients and healthy subjects. Spearman correlation tests were used to determine the association between IgG1 and IgG3 reactivity to apoptotic cells and C4d binding on target cells, between IgG concentration before and after purification, between IgG reactivity to apoptotic cells and patient age and between IgG reactivity to wild type Jurkat cells and reactivity to class I negative Jurkat cells. Kidney graft loss post-transplant was reported as death censored graft loss. Graft-survival rates were computed using the Kaplan-Meier method and groups were compared by the log-rank test. Cox models were used to adjust the effects of IgG reactivity to apoptotic cells for potentially confounding factors including reactivity to HLA antibodies, sex, age, race, calculated panel reactive antibody (cPRA), delayed graft function (DGF), HLA mismatch, cold ischemia time, induction therapy and living donor transplants. Hazard ratio and 95% confidence interval are provided as measures of strength of association and precision, respectively. All tests were two-sided and a P-value of <0.05 was considered to be statistically significant. General data analysis was conducted using SAS Analytics Software (SAS Institute Inc, Cary, N.C.) and GraphPad Prism (GraphPad Software 4.0, San Diego, Calif.).

### Example 2

### Serum Reactivity to Apoptotic Cells Before Transplantation

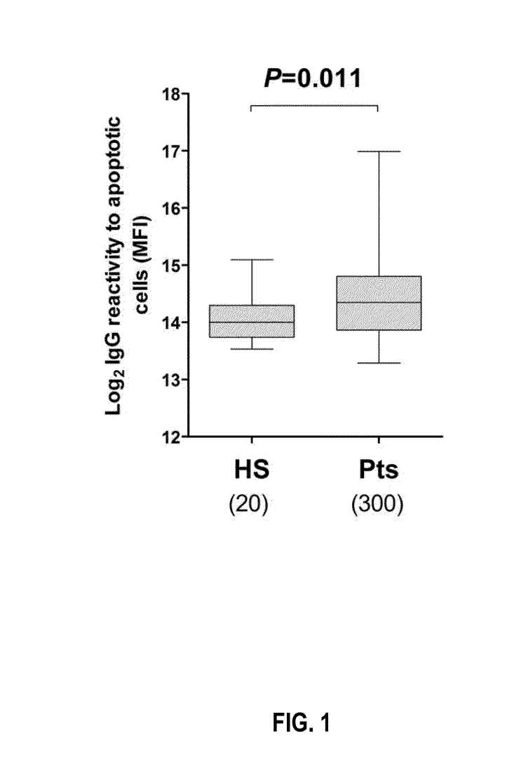

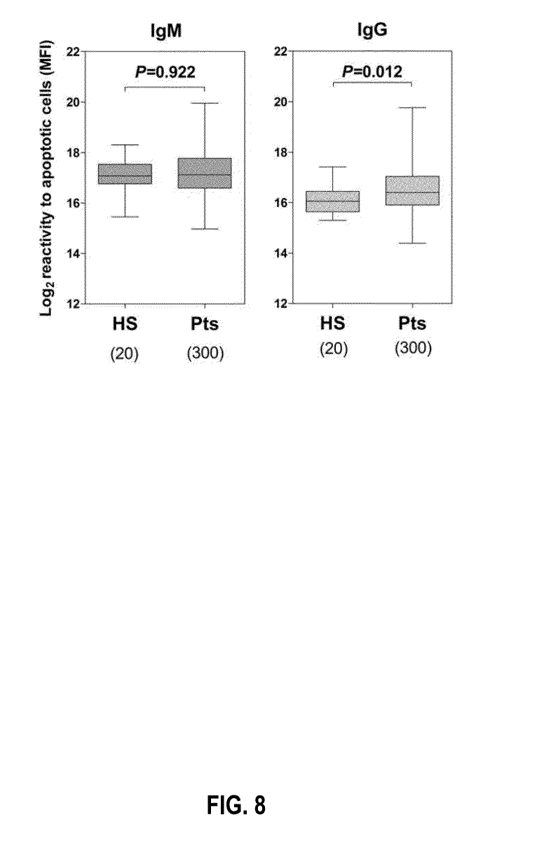

In order to evaluate the contribution of anti-apoptotic cell antibodies to pre-sensitization, pre-transplant serum IgG reactivity was assessed compared to apoptotic cells in 300 kidney transplant recipients as well as 20 healthy controls. The binding of IgG to apoptotic cells can be masked by serum proteins (30). Serum IgG was purified before assessing their reactivity to apoptotic cells. As shown in FIG. 1, purified IgG reactivity to apoptotic cells was significantly higher in pre-transplant serum compared to healthy subjects (P=0.011). Comparable results were obtained when assessing non purified serum IgG reactivity to apoptotic cells (FIG. 8). In contrast, no significant difference in IgM reactivity to apoptotic cells was observed between pre-transplant patients and healthy subjects (P=0.922, FIG. 8).

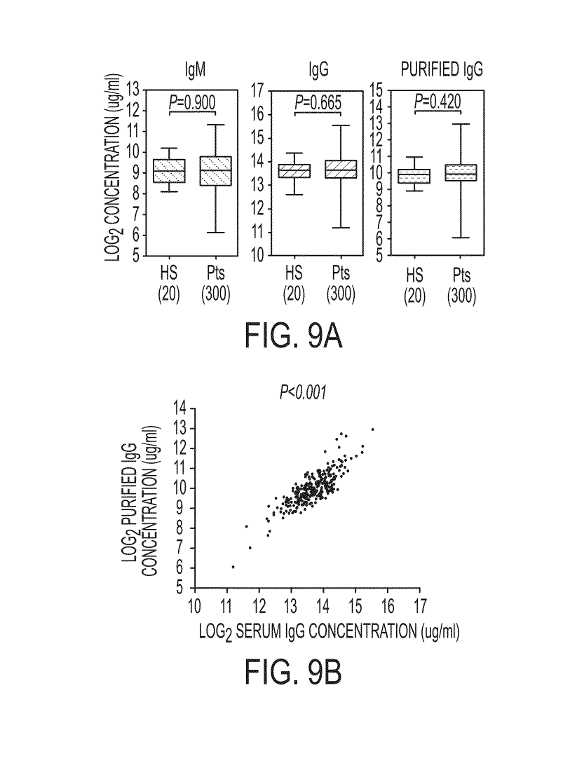

The difference observed in reactivity to apoptotic cells was verified between the two groups and was not solely due to serum immunoglobulin levels. As illustrated in FIG. 9A, concentrations of serum IgM, IgG as well as purified IgG were not significantly different between pre-transplant patients and healthy subjects (P=0.900, P=0.665, P=0.420, respectively). Additionally, concentrations of purified IgG were comparable to that of unpurified serum IgG concentrations in all 300 pre-transplant patients (P<0.001, FIG. 9B).

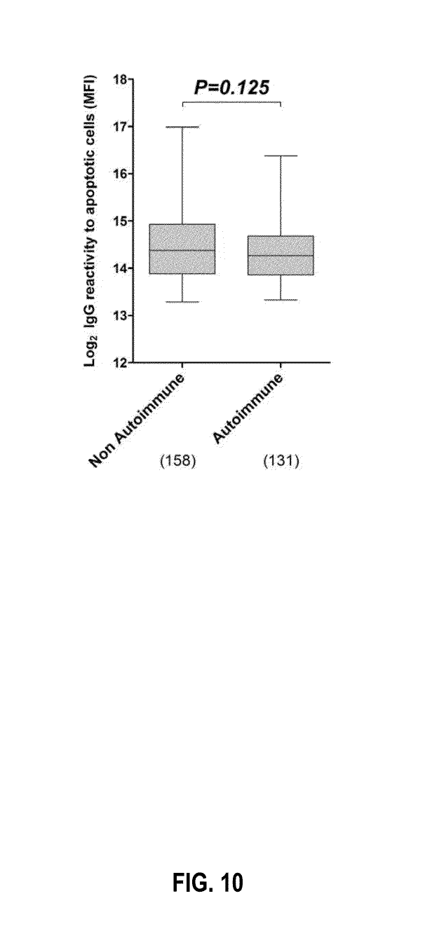

Pre-transplant IgG reactivity to apoptotic cells was then examined and was then determined whether associated with discrete patient characteristics. A positive correlation between age and purified IgG reactivity to apoptotic cells (P=0.024, not shown) was observed. However, reactivity of purified IgG to apoptotic cells did not appear to significantly correlate with sex, race, donor, etiology of kidney failure, previous transplants or history of blood transfusions. No significant difference between patients with autoimmune diseases (including primary focal segmental glomerulosclerosis, IgA nephropathy, type I diabetes, systemic lupus erythematosus and immune complex diseases) and patients with non-autoimmune diseases (FIG. 10) was observed.

### Example 3

### Serum Reactivity to HLA Class I Negative Jurkat Cells and Viable Jurkat Cells

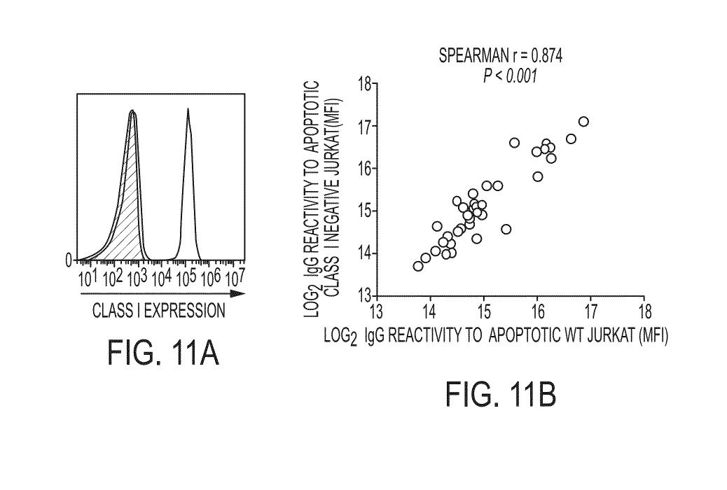

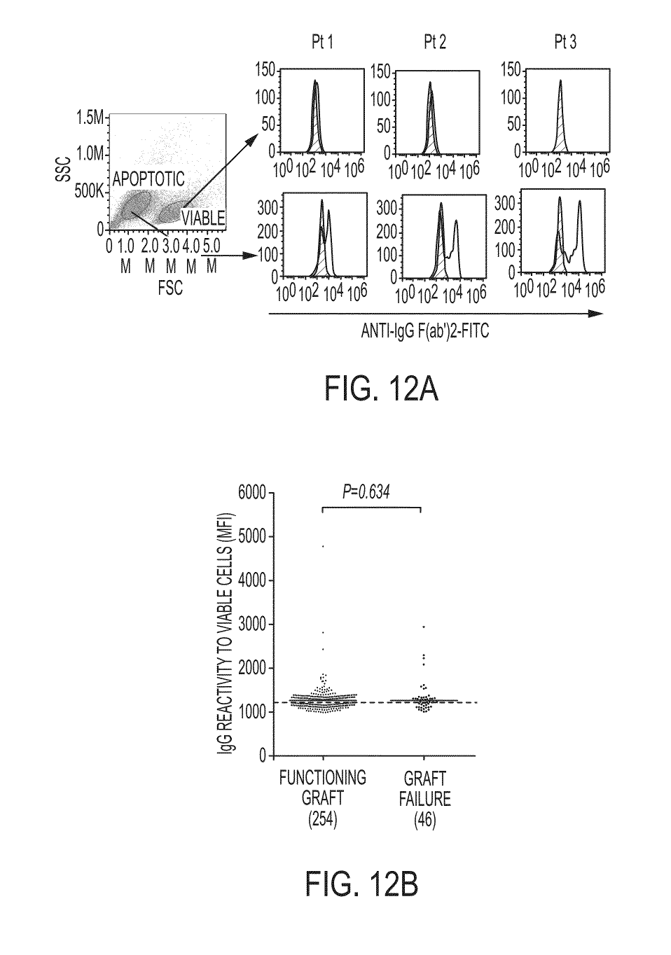

To ensure that reactivity to apoptotic Jurkat cells was not due to the recognition of HLA class I, class I negative Jurkat cells were generated through β-2 microglobulin knockdown using shRNA transfection to use as target (FIG. 11A). As shown in FIG. 11B, the binding of purified IgG to apoptotic class I negative cells is comparable to that of wild type Jurkat cells in the 39 patients with high IgG reactivity to HLA class I (MFI>1000), (r=0.874, P<0.001), indicating that these antibodies recognize other antigenic structures than HLA on apoptotic cells. Lastly, as illustrated in FIG. 12A for 3 representative pre-transplant samples, no reactivity was detected on viable Jurkat cells.

### Example 4

### Pre-Transplant Purified IgG Reactivity to Apoptotic Cells and Kidney Graft Survival

The mean duration of follow-up for all patients included in this retrospective study was 81.2±35.3 months. Forty-six patients lost their grafts and returned to dialysis due to various complications. The causes of graft loss are reported in Table 2.

TABLE 2
Cause of graft loss (N = 46)

| Cause of graft loss | Number |
|---|---|
| Antibody mediated rejection | 17 |
| Antibody mediated rejection & Cellular rejection | 6 |
| Cellular rejection | 4 |
| BK nephropathy | 5 |
| Recurrent IgA nephropathy | 3 |
| Recurrent FSGS | 4 |
| Transplant glomerulopathy | 7 |

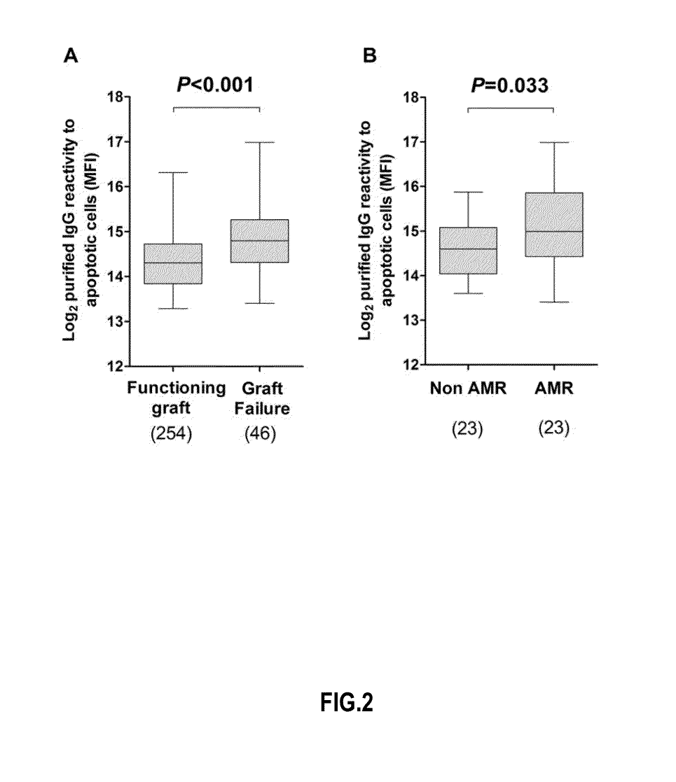

As depicted in FIG. 2A, these patients had significantly higher purified IgG reactivity to apoptotic cells before transplantation compared to those with functioning graft (P<0.001). In contrast, pre-transplant IgG reactivity to viable cells was not significantly different between patients with functioning graft and patients who experienced graft loss (P=0.634, FIG. 12B). Remarkably, among the 46 patients who lost their grafts, pre-transplant purified IgG reactivity to apoptotic cells was significantly increased in those whose graft loss was attributed to AMR compared to patients with other causes of graft loss (P=0.033, FIG. 2B).

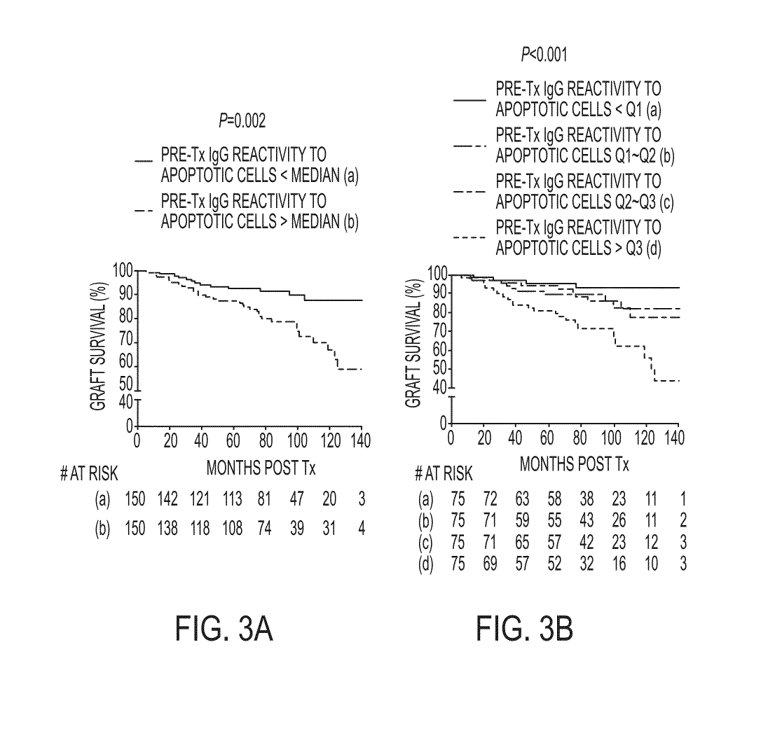

FIG. 3A reports the death with functioning graft censored Kaplan-Meier survival outcome for patients with pre-transplant purified IgG reactivity to apoptotic cells above or below the median value (P=0.002). The patients were separated into 4 groups according to their reactivity to apoptotic cells: below the 1^{st} quartile, between the 1^{st} quartile and the 2^{nd} quartile, between the 2^{nd} quartile and the 3^{rd} quartile or above the 3^{rd} quartile value. As shown in this figure, the graft survival rate was significantly different between these groups (P<0.001, FIG. 3B). Patients with pre-transplant purified IgG reactivity to apoptotic cells above the 3^{rd} quartile experienced the worst outcome while patients whose purified IgG reactivity to apoptotic cells was below the 1^{st} quartile value had the highest graft survival rate. The influence of IgG reactivity to apoptotic cells on graft survival was only noticeable after approximately 1 year post-transplantation as apparent in the graft survival curve.

That the effect on graft survival was only visible after approximately 1 year post-transplantation is consistent with results reported by Opelz who showed that the effect of high PRA measurements on graft survival was only apparent after one year post-transplant. The current results suggest that anti-apoptotic cell IgG may have contributed to sensitization in these patients.

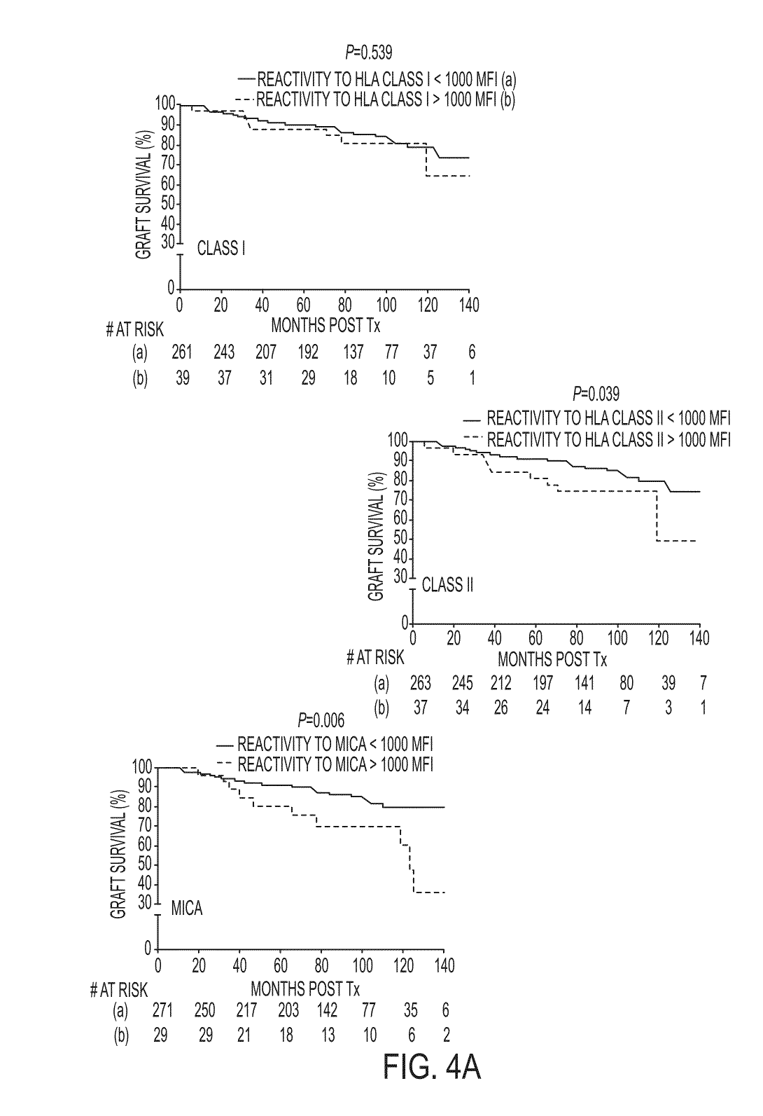

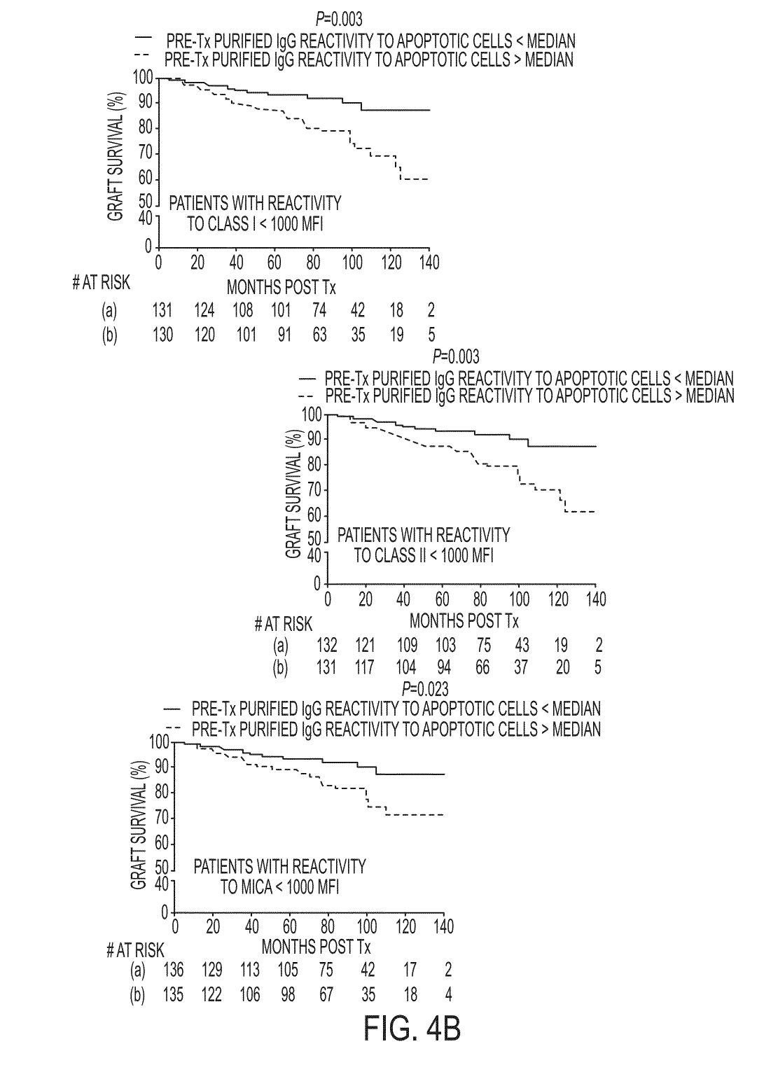

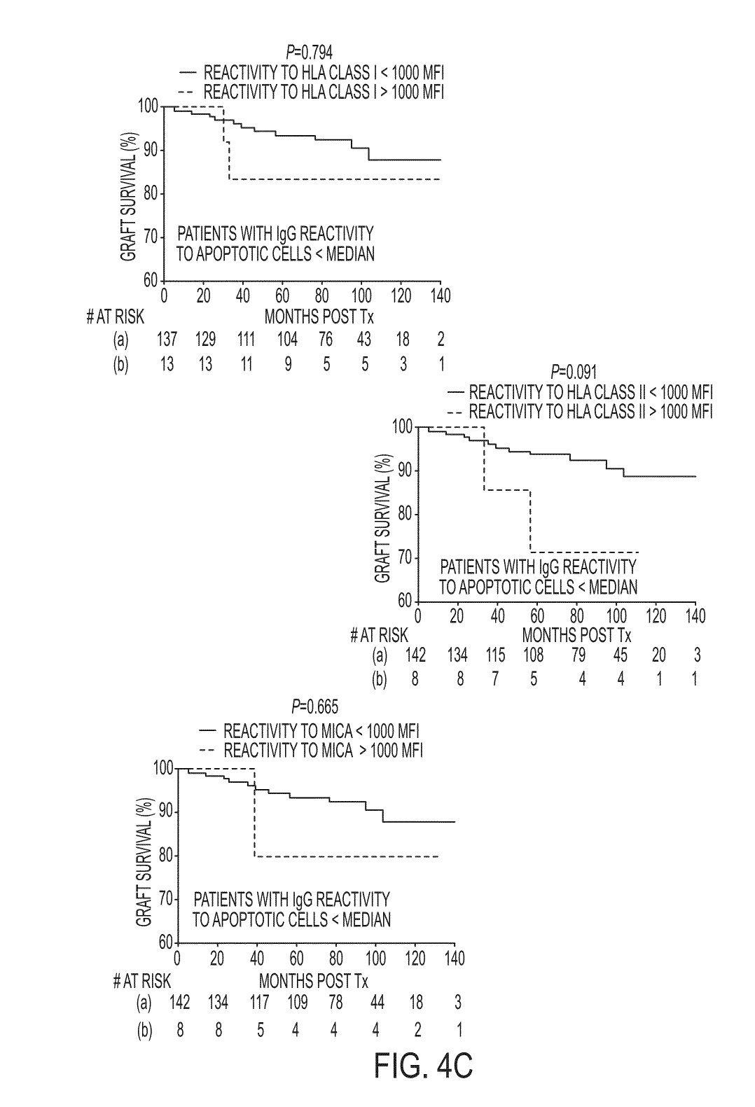

None of the 300 patients had detectable DSA at time of transplant, however, the overall pre-transplant serum reactivity to mixed HLA class I, II and MICA by Luminex was assessed in order to evaluate its effect on the graft outcome. Results showed that pre-existing reactivity to HLA class II and MICA but not HLA class I above a cutoff value of 1000 MFI was associated with decreased graft survival (P=0.039, P=0.006, P=0.539, respectively; FIG. 4A). Whether reactivity to apoptotic cells still correlated with graft loss was investigated when patients with reactivity to HLA class I, class II or MICA above 1000 MFI were excluded. As shown in FIG. 4B, pre-transplant IgG reactivity to apoptotic cells still affected graft outcome in patients with low reactivity to HLA class I, class II and MICA (P=0.003, P=0.003, P=0.023, respectively). Conversely, we examined whether reactivity to HLA class I, class II and MICA influenced the graft outcome in patients with IgG reactivity to apoptotic cells below the median value. As depicted in FIG. 4C, pre-existing HLA class I, class II and MICA reactive antibodies did not correlate with graft loss in patients with IgG reactivity to apoptotic cells below the median value (P=0.794, P=0.091, P=0.665, respectively). Of note, the number of patients included in this latter analysis was limited. To rule out the possibility that the risk-elevation for graft loss with increased levels of pre-transplant IgG reactivity to apoptotic cells was an artifact due to confounding with other factors including other IgG reactivities, sex, age, race, cPRA, DGF, HLA mismatch, cold ischemia time, induction therapy and living donor transplants, a Cox proportional hazards analysis was performed including these covariates. The results confirmed that IgG reactivity to apoptotic cells as an explanatory factor remained significantly associated to graft loss even when the reactivity to HLA class I, class II and MICA as well as other variables were included and adjusted for in the statistical model (Table 3).

TABLE 3
Cox proportional hazards model. Hazard ratio (HR) with 95%
confidence interval (95% CI) for graft survival
in accordance with different variables

| | Hazard | 95% confidence | |
|---|---|---|---|
| Variable | ratio | interval | p-value |
| IgG reactivity to apoptotic cells^{a} | 2.271 | 1.530~3.369 | <0.001 |
| IgG reactivity to HLA class I^{a} | 0.909 | 0.739~1.118 | 0.366 |
| IgG reactivity to HLA class II^{a} | 1.212 | 0.979~1.501 | 0.077 |
| IgG reactivity to MICA^{a} | 1.015 | 0.863~1.195 | 0.857 |
| Sex (male vs female) | 0.964 | 0.499~1.865 | 0.915 |
| African indicator | 1.209 | 0.481~3.040 | 0.687 |
| Asians indicator | 0.600 | 0.079~4.539 | 0.621 |
| Hispanic indicator | 2.062 | 0.683~6.220 | 0.199 |
| Age | 0.996 | 0.972~1.019 | 0.715 |
| cPRA | 0.993 | 0.970~1.017 | 0.574 |
| DGF | 1.981 | 0.851~4.611 | 0.113 |
| HLA mismatch | 1.227 | 0.977~1.541 | 0.079 |
| Cold ischemia time | 1.000 | 0.947~1.057 | 0.994 |
| Induction therapy | 0.692 | 0.326~1.470 | 0.338 |
| Living related donor | 0.659 | 0.205~2.122 | 0.484 |
| Living unrelated donor | 1.029 | 0.372~2.846 | 0.956 |

^{a}per unit increase in log₂ (1 + IgG reactivity)

In additional Cox models not shown, the hazard ratio for IgG reactivity to apoptotic cells remained constant throughout post-transplant follow-up time.

In some embodiments the generation of de novo DSA and non-donor-specific antibodies (NDSA) post-transplant is evaluated along with post-transplant levels of anti-apoptotic IgG, to predict a higher risk of transplant rejection.

### Example 4

### Subclasses of Serum IgG Reactive to Apoptotic Cells

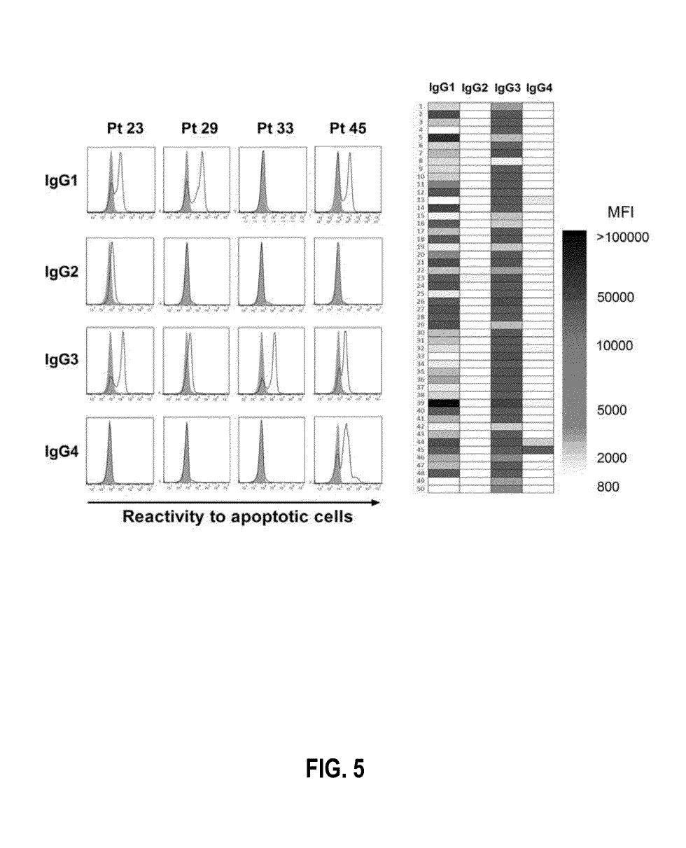

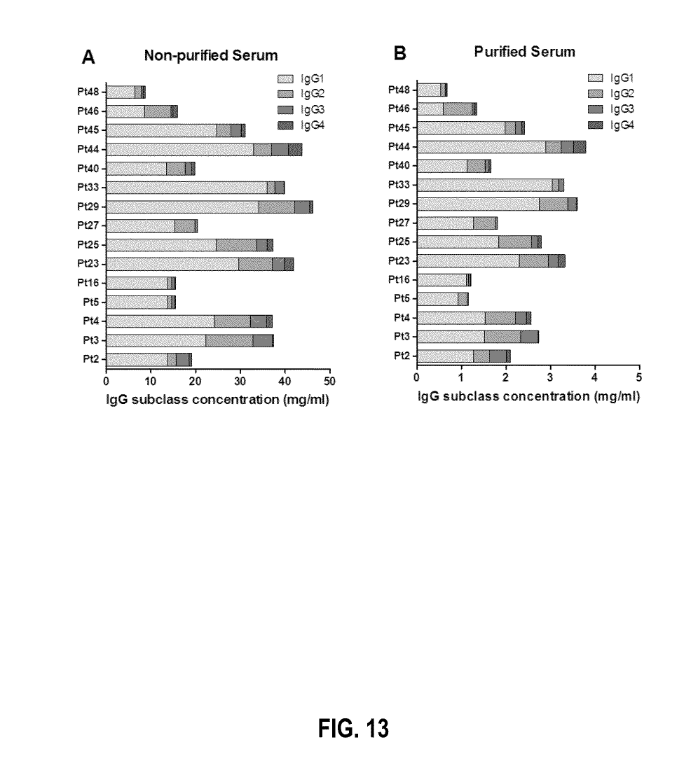

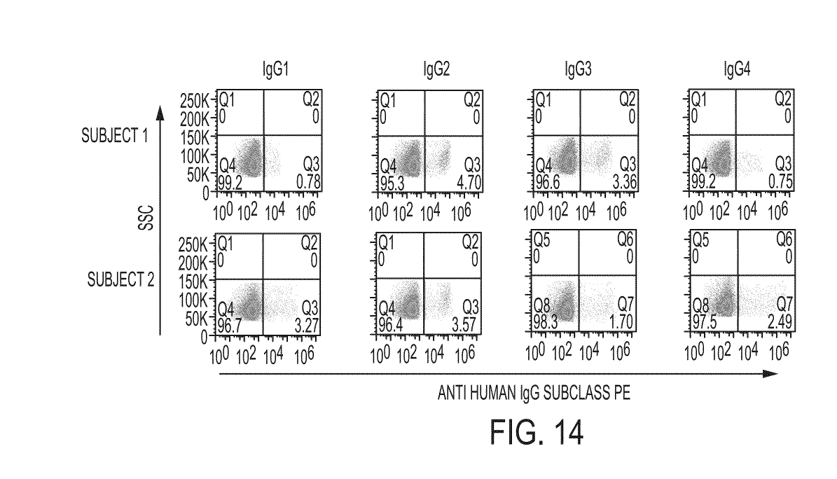

The activation of the complement pathway is a primary effector function of antibodies implicated in graft failure (32-34). It was investigated whether IgG reactive to apoptotic cells could also activate complement. Distinct secondary antibodies were used in flow cytometry experiments to determine the subclasses of IgG reactive to apoptotic cells. These experiments were conducted using IgG purified from the 50 most reactive serum specimens. For all specimens the purified serum IgG reactivity to apoptotic cells was almost exclusively mediated by complement fixing IgG1 or IgG3 subclasses or a combination of the two (FIG. 5). Since only few samples showed IgG4 or IgG2 reactivity to apoptotic cells, it was verified that the purification procedure did not eliminate these two IgG subclasses. As shown in FIG. 13, the concentrations of all four IgG subclasses were comparable before and after IgG purification. We also tested the reactivity of all anti-IgG subclass secondary antibodies used in these assays on peripheral blood B cells from 2 healthy subjects. A distinct positive subset was detectable for each anti-IgG subclass as depicted in FIG. 14.

### Example 5

### IgG1 and IgG3 Reactive to Apoptotic Cells Activate Complement

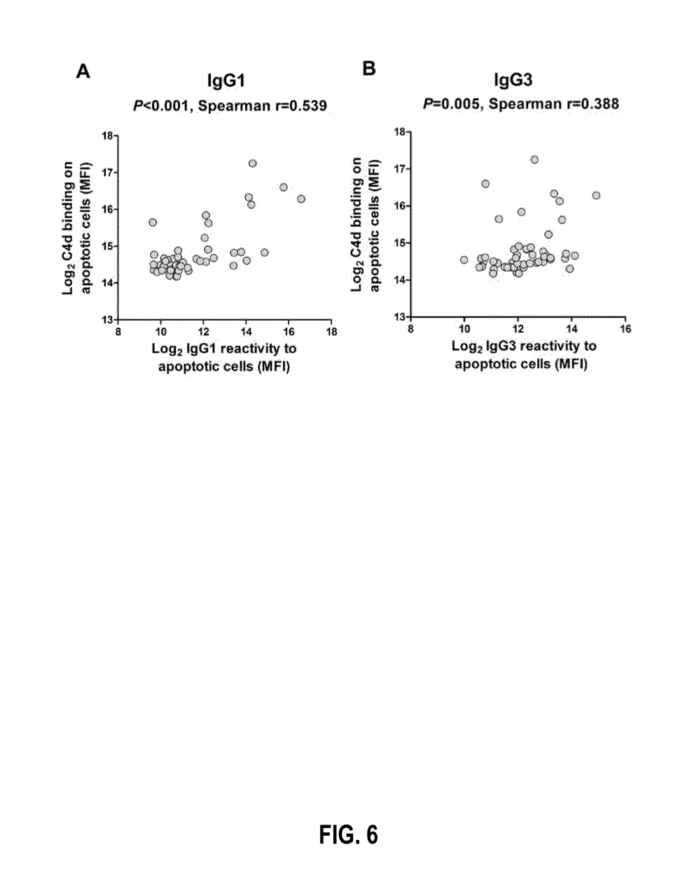

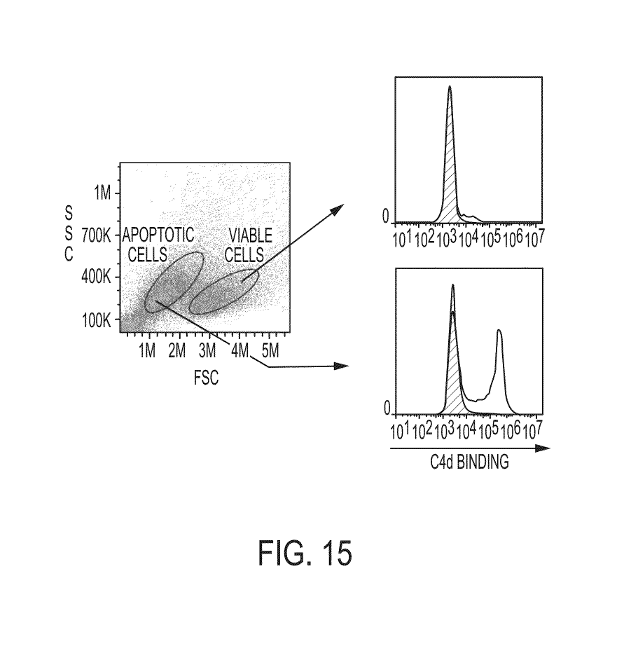

The implication of the complement pathway in graft dysfunction is commonly revealed by the deposition of C4d into the allograft tissue (35-38). An in vitro assay was used to assess whether IgG reactive to apoptotic cells purified from pre-transplant serum specimens have the capacity to activate complement, resulting in the deposition of C4d on target cells (FIG. 15). As expected, purified IgG comprising primarily complement fixing IgG1 and IgG3 had the capacity to activate complement. Moreover, we observed a strong association between IgG1 and IgG3 reactivity to apoptotic cells and C4d deposition on target cells (P<0.001 and P=0.005, respectively; FIG. 6).

It is still unclear as to what antigens are recognized by these anti-apoptotic IgG antibodies at the surface of apoptotic cells. In a previous study, it was reported that the characterization of monoclonal antibodies reactive to apoptotic cells established from a patient with AMR (30). These monoclonal antibodies were all polyreactive, i.e. they reacted to multiple antigens as diverse as DNA or lipopolysaccharide. It was also found that 2 of 6 polyreactive antibodies bound equally to phosphatidylserine and lysophosphatidylcholine. Both antigens are known immunogenic structures exposed at the surface of apoptotic cells. These results indicate that polyreactive antibodies can recognize distinct antigens on apoptotic cells. The fact that anti-apoptotic cell IgG is almost exclusively composed of complement fixing IgG1 and IgG3 is novel and intriguing. It is noteworthy however that both IgG1 and IgG3 have complement activating properties. Late allograft failures often involve complement activation, as revealed by the deposition of C4d on the graft tissue. IgG1 and IgG3 appear to play a dominant role in this process (44-47). Without being bound by theory, the view is that IgG1 and IgG3 reactive to apoptotic cells participate in the overall inflammatory response to the allograft and eventually contribute to graft loss.

### REFERENCES

1. Terasaki P I. Humoral theory of transplantation. Am J Transplant 2003; 3(6):665-673.
2. Jordan S C, Pescovitz M D. Presensitization: the problem and its management. Clin J Am Soc Nephrol 2006; 1(3):421-432.
3. Susal C, Opelz G. Kidney graft failure and presensitization against HLA class I and class II antigens. Transplantation 2002; 73(8):1269-1273.
4. Susal C, Dohler B, Opelz G. Presensitized kidney graft recipients with HLA class I and II antibodies are at increased risk for graft failure: a Collaborative Transplant Study report. Hum Immunol 2009; 70(8):569-573.
5. Susal C, Dohler B, Sadeghi M, Ovens J, Opelz G. HLA antibodies and the occurrence of early adverse events in the modern era of transplantation: a collaborative transplant study report. Transplantation 2009; 87(9): 1367-1371.
6. Amico P, Honger G, Mayr M, Steiger J, Hopfer H, Schaub S. Clinical relevance of pretransplant donor-specific HLA antibodies detected by single-antigen flow-beads. Transplantation 2009; 87(11): 1681-1688.
7. Zou Y, Stastny P, Susal C, Dohler B, Opelz G. Antibodies against MICA antigens and kidney-transplant rejection. N Engl J Med 2007; 357(13):1293-1300.
8. Dragun D. Humoral responses directed against non-human leukocyte antigens in solid-organ transplantation. Transplantation 2008; 86(8):1019-1025.
9. Sumitran-Holgersson S. Relevance of MICA and other non-HLA antibodies in clinical transplantation. Curr Opin Immunol 2008; 20(5):607-613.
10. Jackson A M, Kuperman M B, Montgomery R A. Multiple hyperacute rejections in the absence of detectable complement activation in a patient with endothelial cell reactive antibody. Am J Transplant 2012; 12(6):1643-1649.
11. Crespo M, Pascual M, Tolkoff-Rubin N, Mauiyyedi S, Collins A B, Fitzpatrick D et al. Acute humoral rejection in renal allograft recipients: I. Incidence, serology and clinical characteristics. Transplantation 2001; 71 (5): 652-658.
12. Opelz G. Non-HLA transplantation immunity revealed by lymphocytotoxic antibodies. Lancet 2005; 365(9470):1570-1576.
13. Solgi G, Furst D, Mytilineos J, Pourmand G, Amirzargar A A. Clinical relevance of pre and post-transplant immune markers in kidney allograft recipients: anti-HLA and MICA antibodies and serum levels of sCD30 and sMICA. Transpl Immunol 2012; 26(2-3):81-87.
14. Dragun D, Muller D N, Brasen J H, Fritsche L, Nieminen-Kelha M, Dechend R et al. Angiotensin II type 1-receptor activating antibodies in renal-allograft rejection. N Engl J Med 2005; 352(6):558-569.
15. Kalache S, Dinavahi R, Pinney S, Mehrotra A, Cunningham M W, Heeger P S. Anticardiac myosin immunity and chronic allograft vasculopathy in heart transplant recipients. J Immunol 2011; 187(2):1023-1030.
16. Joosten S A, Sijpkens Y W, van Ham V, Trouw L A, van der Vlag J, van den Heuvel B et al. Antibody response against the glomerular basement membrane protein agrin in patients with transplant glomerulopathy. Am J Transplant 2005; 5(2):383-393.
17. Rose M L. Role of anti-vimentin antibodies in allograft rejection. Hum Immunol 2013; 74(11):1459-1462.
18. Linke A T, Marchant B, Marsh P, Frampton G, Murphy J, Rose M L. Screening of a HUVEC cDNA library with transplant-associated coronary artery disease sera identifies RPL7 as a candidate autoantigen associated with this disease. Clin Exp Immunol 2001; 126(1):173-179.
19. Porcheray F, DeVito J, Yeap B Y, Xue L, Dargon I, Paine R et al. Chronic humoral rejection of human kidney allografts associates with broad autoantibody responses. Transplantation 2010; 89(10): 1239-1246.
20. Warraich R S, Pomerance A, Stanley A, Banner N R, Dunn M J, Yacoub M H. Cardiac myosin autoantibodies and acute rejection after heart transplantation in patients with dilated cardiomyopathy. Transplantation 2000; 69(8): 1609-1617.
21. Wheeler C H, Collins A, Dunn M J, Crisp S J, Yacoub M H, Rose M L. Characterization of endothelial antigens associated with transplant-associated coronary artery disease. J Heart Lung Transplant 1995; 14(6 Pt 2):788-197.
22. Cristol J P, Vela C, Maggi M F, Descomps B, Mourad G. Oxidative stress and lipid abnormalities in renal transplant recipients with or without chronic rejection. Transplantation 1998; 65(10):1322-1328.
23. Cardinal H, Dieude M, Brassard N, Qi S, Patey N, Soulez M et al. Antiperlecan antibodies are novel accelerators of immune-mediated vascular injury. Am J Transplant 2013; 13(4):861-874.
24. Avrameas S. Natural autoantibodies: from ‘honor autotoxicus’ to ‘gnothi seauton’. Immunol Today 1991; 12(5):154-159.
25. Ehrenstein M R, Notley C A. The importance of natural IgM: scavenger, protector and regulator. Nat Rev Immunol 2010; 10(11):778-786.
26. Fu M, Fan P S, Li W, Li C X, Xing Y, An J G et al. Identification of poly-reactive natural IgM antibody that recognizes late apoptotic cells and promotes phagocytosis of the cells. Apoptosis: an international journal on programmed cell death 2007; 12(2):355-362.
27. Silverman G J, Gronwall C, Vas J, Chen Y. Natural autoantibodies to apoptotic cell membranes regulate fundamental innate immune functions and suppress inflammation. Discov Med 2009; 8(42):151-156.
28. Peng Y, Kowalewski R, Kim S, Elkon K B. The role of IgM antibodies in the recognition and clearance of apoptotic cells. Mol Immunol 2005; 42(7):781-787.
29. Porcheray F, DeVito J, Helou Y, Dargon I, Fraser J W, Nobecourt P et al. Expansion of polyreactive B cells cross-reactive to HLA and self in the blood of a patient with kidney graft rejection. Am J Transplant 2012; 12(8):2088-2097.
30. Porcheray F, Fraser J W, Gao B, McColl A, Devito J, Dargon I et al. Polyreactive antibodies developing amidst humoral rejection of human kidney grafts bind apoptotic cells and activate complement. Am J Transplant 2013; 13(10):2590-2600.
31. Bellucci R, Nguyen H N, Martin A, Heinrichs S, Schinzel A C, Hahn W C et al. Tyrosine kinase pathways modulate tumor susceptibility to natural killer cells. J Clin Invest 2012; 122(7):2369-2383.
32. Colvin R B. Antibody-mediated renal allograft rejection: diagnosis and pathogenesis. J Am Soc Nephrol 2007; 18(4):1046-1056.
33. Stegall M D, Chedid M F, Cornell L D. The role of complement in antibody-mediated rejection in kidney transplantation. Nat Rev Nephrol 2012; 8(11):670-678.
34. Fukuda M. Evaluation and clinical significance of circulating immune complexes after renal transplantation. Transplantation 1983; 36(2):155-160.
35. Bohmig G A, Exner M, Habicht A, Schillinger M, Lang U, Kletzmayr J et al. Capillary C4d deposition in kidney allografts: a specific marker of alloantibody-dependent graft injury. J Am Soc Nephrol 2002; 13(4):1091-1099.
36. Magil A B, Tinckam K. Monocytes and peritubular capillary C4d deposition in acute renal allograft rejection. Kidney Int 2003; 63(5):1888-1893.
37. Regele H, Bohmig G A, Habicht A, Gollowitzer D, Schillinger M, Rockenschaub S et al. Capillary deposition of complement split product C4d in renal allografts is associated with basement membrane injury in peritubular and glomerular capillaries: a contribution of humoral immunity to chronic allograft rejection. J Am Soc Nephrol 2002; 13(9):2371-2380.
38. Lorenz M, Regele H, Schillinger M, Exner M, Rasoul-Rockenschaub S, Wahrmann M et al. Risk factors for capillary C4d deposition in kidney allografts: evaluation of a large study cohort. Transplantation 2004; 78(3):447-452.
39. Liu S, Cerutti A, Casali P, Crow M K. Ongoing immunoglobulin class switch DNA recombination in lupus B cells: analysis of switch regulatory regions. Autoimmunity 2004; 37(6-7):431-443.
40. Mietzner B, Tsuiji M, Scheid J, Velinzon K, Tiller T, Abraham K et al. Autoreactive IgG memory antibodies in patients with systemic lupus erythematosus arise from nonreactive and polyreactive precursors. Proc Natl Acad Sci USA 2008; 105(28):9727-9732.
41. Jofre R, Rodriguez-Benitez P, Lopez-Gomez J M, Perez-Garcia R. Inflammatory syndrome in patients on hemodialysis. J Am Soc Nephrol 2006; 17(12 Suppl 3):5274-280.
42. Carrero J J, Qureshi A R, Axelsson J, Avesani C M, Suliman M E, Kato S et al. Comparison of nutritional and inflammatory markers in dialysis patients with reduced appetite. Am J Clin Nutr 2007; 85(3):695-701.
43. Zhang J, Jacobi A M, Wang T, Berlin R, Volpe B T, Diamond B. Polyreactive autoantibodies in systemic lupus erythematosus have pathogenic potential. J Autoimmun 2009; 33(3-4):270-274.
44. Colvin R B, Smith R N. Antibody-mediated organ-allograft rejection. Nat Rev Immunol 2005; 5(10):807-817.
45. Bruggemann M, Williams G T, Bindon C I, Clark M R, Walker M R, Jefferis R et al. Comparison of the effector functions of human immunoglobulins using a matched set of chimeric antibodies. J Exp Med 1987; 166(5):1351-1361.
46. Michaelsen T E, Garred P, Aase A. Human IgG subclass pattern of inducing complement-mediated cytolysis depends on antigen concentration and to a lesser extent on epitope patchiness, antibody affinity and complement concentration. Eur J Immunol 1991; 21(1):11-16.
47. Lucisano Valim Y M, Lachmann P J. The effect of antibody isotype and antigenic epitope density on the complement-fixing activity of immune complexes: a systematic study using chimaeric anti-NIP antibodies with human Fc regions. Clin Exp Immunol 1991; 84(1):1-8.

What is claimed is:

1. A method for predicting transplant rejection in a pre-transplant subject or a subject who has had a transplant,
(a) obtaining a biological sample from the subject and a biological sample from a group of healthy control subjects;
(b) isolating IgG antibodies from the subject and from at least three control samples;
(c) contacting a test population of apoptotic cells with the subject IgG antibodies and contacting at least three control populations of apoptotic cells with the IgG antibodies from each of the control samples, for a time and under conditions that permit the antibodies to bind to the apoptotic cells; and
(d) determining the amount of binding of the IgG antibodies to apoptotic cells in the test population and in the control populations, and if the amount of IgG antibody binding in the test population is higher than the median value+2 standard deviation of three control specimens, then determining that the subject is at a high risk of transplant rejection.

2. The method of claim 1, wherein the apoptotic cells do not express HLA class 1 antigens.

3. The method of claim 1, wherein the apoptotic cells are selected from the group consisting of Jurkat cells, 293 Human Embryonic Kidney cells, and endothelial cells including human umbilical cord endothelial cells.

4. The method of claim 1, wherein determining the amount of binding of the IgG antibodies to apoptotic cells in the test population and in the control populations comprises incubating the test and control apoptotic cells of step (e) with a secondary anti-IgG antibody; and
assessing binding of the IgG antibodies to quantitate antibody binding.

5. The method of claim 1 wherein the subject is human.

6. The method of claim 1 wherein the pre-transplant subject is in need of a tissue transplant selected from the group consisting of corneas, bone, tendons, ligaments, heart valves, skin, blood vessels, veins, arteries and hematopoietic stem cell transplants

7. The method of claim 1 wherein the pre-transplant subject is in need of an organ transplant wherein the organ is selected from the group consisting of pancreas, heart, kidney, lung, liver, bladder and intestine.

8. The method of claim 7, wherein the organ is a kidney.

9. The method of claim 1, wherein the biological sample is selected from the group consisting of blood, plasma, serum or other blood derived products, csf, synovial fluid, bronchioalveolar lavage and ascites.

10. The method of claim 9, wherein the biological sample is serum.

11. The method of claim 1, wherein the IgG antibodies are purified.

12. The method of claim 1, wherein the apoptotic cells are Jurkat T cell leukemia cells.

13. The method of claim 1, wherein apoptosis is induced by UV light, biological or chemical means.

14. The method of claim 13, wherein apoptosis is induced by a method selected from the group consisting of activating either Fas or TNF-receptors with agonist antibody, or administering doxorubicin, 5-fljorouracil, paclitaxel, vinblastine, staurosporine, or UV light.

15. The method of claim 1, wherein apoptosis is induced by incubation with anti-FAS antibody or exposure to UV light.

16. The method of claim 1, further comprising
(e) if it is determined that the subject is at risk of transplantation rejection, then administering to the subject a desensitization treatment.

17. The method of claim 16, wherein the desensitization treatment comprises administering a therapeutically effective amount of an immunosuppressant drug selected from the group consisting of Bortezomib, cyclosporine, rapamycin, Campath I, thymoglobulin, (rATG), anti-thymocytic antibody, Rituximab, and Gamimune N, dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur, fluocinolone, triaminolone, anecortave acetate, flurometholone, medrysone, IVIG and prednislone.

18. The method of claim 16, wherein the desensitization treatment comprises plasmapheresis.

19. A kit for prediction of rejection of a transplanted organ or tissue comprising:
(a) a biological sample collection device to obtain a serum sample from a subject;
(b) a serum sample from each of at least 3 normal control subjects comprising isolated IgG antibodies;
(c) and instructions for using the kit.

20. The kit of claim 19, further comprising
(d) apoptotic cells capable of binding IgG antibodies.

21. The method of claim 1, wherein amount of IgG antibodies in the samples is determined using an immunosorbent assay using an antibody directed to anti-apoptotic IgG that is conjugated to a label or enzyme, flow cytometry, or ELISA.

22. The method of claim 1, wherein the IgG antibody reactivity to apoptotic cells is mediated by complement fixing IgG1 or IgG3 or a combination of the two.
